SEQUENCE LISTING (33 sequences)

SEQ ID NO: 1 - DNA, 70 bp, Artificial Sequence
  synthetic sequence
gtcgtgctcaagacgcactacgacgtttaaaccttaattaagcatggtctcgggctgggacttccacccg

SEQ ID NO: 2 - DNA, 70 bp, Artificial Sequence
  Synthetic Sequence
cgggtggaagtcccagcccgagaccatgcttaattaaggtttaaacgtcgtagtgcgtcttgagcacgac

SEQ ID NO: 3 - DNA, 100 bp, Artificial Sequence
  synthetic sequence
aagattggcccgtcgattgtcgtgctcaagacgcactacgacgtttaaaccttaattaagcatggtctcgggctgggacttccacccggagacgggcacg

SEQ ID NO: 4 - DNA, 100 bp, Artificial Sequence
  synthetic sequence
cgtgcccgtctccgggtggaagtcccagcccgagaccatgcttaattaaggtttaaacgtcgtagtgcgtcttgagcacgacaatcgacgggccaatctt

SEQ ID NO: 5 - DNA, 200 bp, Artificial Sequence
  synthetic sequence
gcctgagcgccgacgtgccgacagcgcgcgagctgctgtacctggccgacaagattggcccgtcgattgtcgtgctcaagacgcactacgacgtttaaaccttaattaagcatggtctcgggctgggacttccacccggagacgggcacgggagcccagctggcgtcgctggcgcgcaagcacggcttcctcatcttcga

SEQ ID NO: 6 - DNA, 200 bp, Artificial Sequence
  synthetic sequence
tcgaagatgaggaagccgtgcttgcgcgccagcgacgccagctgggctcccgtgcccgtctccgggtggaagtcccagcccgagaccatgcttaattaaggtttaaacgtcgtagtgcgtcttgagcacgacaatcgacgggccaatcttgtcggccaggtacagcagctcgcgcgctgtcggcacgtcggcgctcaggc

SEQ ID NO: 7 - DNA, 330 bp, Artificial Sequence
  synthetic sequence
gcggccaggagcgagacggcgacgcacccgctgacggcttacctgttcaagctcatggacctcaaggcgtccaacctgtgcctgagcgccgacgtgccgacagcgcgcgagctgctgtacctggccgacaagattggcccgtcgattgtcgtcgtttaaaccttaattaagtctcgggctgggacttccacccggagacgggcacgggagcccagctggcgtcgctggcgcgcaagcacggcttcctcatcttcgaggaccgcaagtttggcgacattggccacaccgtcgagctgcagtacacgggcgggtcggcgcgcatcatcgact

SEQ ID NO: 8 - DNA, 730 bp, Artificial Sequence
  synthetic sequence
ctactcctcttcctcccgcacggctgtcgcgcttccaacgtcattcgcacagcagaattgtgccatctctctcttttttttccccccctctaaaccgccacaacggcaccctaagggttaaactatccaaccagccgcagcctcagcctctctcagcctcatcagccatggcaccacacccgacgctcaaggccaccttcgcggccaggagcgagacggcgacgcacccgctgacggcttacctgttcaagctcatggacctcaaggcgtccaacctgtgcctgagcgccgacgtgccgacagcgcgcgagctgctgtacctggccgacaagattggcccgtcgattgtcgtcgtttaaaccttaattaagtctcgggctgggacttccacccggagacgggcacgggagcccagctggcgtcgctggcgcgcaagcacggcttcctcatcttcgaggaccgcaagtttggcgacattggccacaccgtcgagctgcagtacacgggcgggtcggcgcgcatcatcgactgggcgcacattgtcaacgtcaacatggtgcccggcaaggcgtcggtggcctcgctggcccagggcgccaagcgctggctcgagcgctacccctgcgaggtcaagacgtccgtcaccgtcggcacgcccaccatggactcgtttgacgacgacgccgactccagggacgccgagcccgccggcgccgtcaacggcatgggc

SEQ ID NO: 9 - DNA, 1100 bp, Artificial Sequence
  synthetic sequence
ctcacccccaaagtcgcaatatcgggtatcgccgccggcattgaatcgccttctccgctagcatcgactactgctgctctgctctcgttgccagcgctgctccctagaattttgaccaggggacgagcccgacattaaagcaactccctcgcctcgagacgactcggatcgcacgaaattctcccaatcgccgacagttcctactcctcttcctcccgcacggctgtcgcgcttccaacgtcattcgcacagcagaattgtgccatctctctcttttttttccccccctctaaaccgccacaacggcaccctaagggttaaactatccaaccagccgcagcctcagcctctctcagcctcatcagccatggcaccacacccgacgctcaaggccaccttcgcggccaggagcgagacggcgacgcacccgctgacggcttacctgttcaagctcatggacctcaaggcgtccaacctgtgcctgagcgccgacgtgccgacagcgcgcgagctgctgtacctggccgacaagattggcccgtcgattgtcgtcgtttaaaccttaattaagtctcgggctgggacttccacccggagacgggcacgggagcccagctggcgtcgctggcgcgcaagcacggcttcctcatcttcgaggaccgcaagtttggcgacattggccacaccgtcgagctgcagtacacgggcgggtcggcgcgcatcatcgactgggcgcacattgtcaacgtcaacatggtgcccggcaaggcgtcggtggcctcgctggcccagggcgccaagcgctggctcgagcgctacccctgcgaggtcaagacgtccgtcaccgtcggcacgcccaccatggactcgtttgacgacgacgccgactccagggacgccgagcccgccggcgccgtcaacggcatgggctccattggcgtcctggacaagcccatctactcgaaccggtccggcgacggccgcaagggcagcatcgtctccatcaccaccgtcacccagcagtacgagtccgtctcctcgccccggttaacaaaggccatcgccgagggcgacgagtcgctcttcccgggcatcgagga

SEQ ID NO: 10 - DNA, 23 bp, Artificial Sequence
  synthetic sequence
gctcaagacgcactacgacatgg

SEQ ID NO: 11 - DNA, 131 bp, Artificial Sequence
  synthetic
cgcgaaattaatacgactcactatagggctcaagacgcactacgacagttttagagctagaaatagcaagttaaaataaggctagtccgttatcaacttgaaaaagtggcaccgagtcggtgctttttagc

SEQ ID NO: 12 - DNA, 26 bp, Artificial Sequence
  primer
ccatcttggctgacgaaaaaggtctg

SEQ ID NO: 13 - DNA, 29 bp, Artificial Sequence
  primer
catgcaaagatacacatcaatcgcagctg

SEQ ID NO: 14 - DNA, 23 bp, Artificial Sequence
  primer
catcgactactgctgctctgctc

SEQ ID NO: 15 - DNA, 23 bp, Artificial Sequence
  primer
atcgcagctggggtacaatcatc

SEQ ID NO: 16 - DNA, 18 bp, Artificial Sequence
  primer
cggcgatggcctttgtta

SEQ ID NO: 17 - DNA, 100 bp, Artificial Sequence
  synthetic sequence
atcagccgtgcgtgtgaccagtgtaaccaactccgaacgaaatgcgtttaaaccttaattaagcgacgggcagcatccgtgcgctcattgcattggtagg

SEQ ID NO: 18 - DNA, 100 bp, Artificial Sequence
  synthetic sequence
cctaccaatgcaatgagcgcacggatgctgcccgtcgcttaattaaggtttaaacgcatttcgttcggagttggttacactggtcacacgcacggctgat

SEQ ID NO: 19 - DNA, 200 bp, Artificial Sequence
  synthetic sequence
ctcgctctgccgtccgcaaaacctcgtcttcagctccggttcgccgccgaatcagccgtgcgtgtgaccagtgtaaccaactccgaacgaaatgcgtttaaaccttaattaagcgacgggcagcatccgtgcgctcattgcattggtaggcttccgctctttctccgatgccggcgatgaggcggacgcttgactgacct

SEQ ID NO: 20 - DNA, 200 bp, Artificial Sequence
  synthetic sequence
aggtcagtcaagcgtccgcctcatcgccggcatcggagaaagagcggaagcctaccaatgcaatgagcgcacggatgctgcccgtcgcttaattaaggtttaaacgcatttcgttcggagttggttacactggtcacacgcacggctgattcggcggcgaaccggagctgaagacgaggttttgcggacggcagagcgag

SEQ ID NO: 21 - DNA, 19 bp, Artificial Sequence
  synthetic sequence
cgtttaaaccttaattaag

SEQ ID NO: 22 - DNA, 20 bp, Artificial Sequence
  synthetic sequence
caactccgaacgaaatgcga

SEQ ID NO: 23 - DNA, 132 bp, Artificial Sequence
  synthetic sequence
cgcgaaattaatacgactcactataggcaactccgaacgaaatgcgagttttagagctagaaatagcaagttaaaataaggctagtccgttatcaacttgaaaaagtggcaccgagtcggtgctttttttaa

SEQ ID NO: 24 - DNA, 29 bp, Artificial Sequence
  primer
ggcttccccgcctttttcccctgcaattc

SEQ ID NO: 25 - DNA, 36 bp, Artificial Sequence
  primer
atgccacccctcttgacaatgaagccagcataagcc

SEQ ID NO: 26 - DNA, 4197 bp, Streptococcus pyogenes
catatggccccaaaaaagaaacgcaaggttatggataaaaaatacagcattggtctggatatcggaaccaacagcgttgggtgggcagtaataacagatgaatacaaagtgccgtcaaaaaaatttaaggttctggggaatacagatcgccacagcataaaaaagaatctgattggggcattgctgtttgattcgggtgagacagctgaggccacgcgtctgaaacgtacagcaagaagacgttacacacgtcgtaaaaatcgtatttgctacttacaggaaattttttctaacgaaatggccaaggtagatgatagtttcttccatcgtctcgaagaatcttttctggttgaggaagataaaaaacacgaacgtcaccctatctttggcaatatcgtggatgaagtggcctatcatgaaaaataccctacgatttatcatcttcgcaagaagttggttgatagtacggacaaagcggatctgcgtttaatctatcttgcgttagcgcacatgatcaaatttcgtggtcatttcttaattgaaggtgatctgaatcctgataactctgatgtggacaaattgtttatacaattagtgcaaacctataatcagctgttcgaggaaaaccccattaatgcctctggagttgatgccaaagcgattttaagcgcgagactttctaagtcccggcgtctggagaatctgatcgcccagttaccaggggaaaagaaaaatggtctgtttggtaatctgattgccctcagtctggggcttaccccgaacttcaaatccaattttgacctggctgaggacgcaaagctgcagctgagcaaagatacttatgatgatgacctcgacaatctgctcgcccagattggtgaccaatatgcggatctgtttctggcagcgaagaatctttcggatgctatcttgctgtcggatattctgcgtgttaataccgaaatcaccaaagcgcctctgtctgcaagtatgatcaagagatacgacgagcaccaccaggacctgactcttcttaaggcactggtacgccaacagcttccggagaaatacaaagaaatattcttcgaccagtccaagaatggttacgcgggctacatcgatggtggtgcatcacaggaagagttctataaatttattaaaccaatccttgagaaaatggatggcacggaagagttacttgttaaacttaaccgcgaagacttgcttagaaagcaacgtacattcgacaacggctccatcccacaccagattcatttaggtgaacttcacgccatcttgcgcagacaagaagatttctatcccttcttaaaagacaatcgggagaaaatcgagaagatcctgacgttccgcattccctattatgtcggtcccctggcacgtggtaattctcggtttgcctggatgacgcgcaaaagtgaggaaaccatcaccccttggaactttgaagaagtcgtggataaaggtgctagcgcgcagtcttttatagaaagaatgacgaacttcgataaaaacttgcccaacgaaaaagtcctgcccaagcactctcttttatatgagtactttactgtgtacaacgaactgactaaagtgaaatacgttacggaaggtatgcgcaaacctgcctttcttagtggcgagcagaaaaaagcaattgtcgatcttctctttaaaacgaatcgcaaggtaactgtaaaacagctgaaggaagattatttcaaaaagatcgaatgctttgattctgtcgagatctcgggtgtcgaagatcgtttcaacgcttccttagggacctatcatgatttgctgaagataataaaagacaaagactttctcgacaatgaagaaaatgaagatattctggaggatattgttttgaccttgaccttattcgaagatagagagatgatcgaggagcgcttaaaaacctatgcccacctgtttgatgacaaagtcatgaagcaattaaagcgccgcagatatacggggtggggccgcttgagccgcaagttgattaacggtattagagacaagcagagcggaaaaactatcctggatttcctcaaatctgacggatttgcgaaccgcaattttatgcagcttatacatgatgattcgcttacattcaaagaggatattcagaaggctcaggtgtctgggcaaggtgattcactccacgaacatatagcaaatttggccggctctcctgcgattaagaaggggatcctgcaaacagttaaagttgtggatgaacttgtaaaagtaatgggccgccacaagccggagaatatcgtgatagaaatggcgcgcgagaatcaaacgacacaaaaaggtcaaaagaactcaagagagagaatgaagcgcattgaggaggggataaaggaacttggatctcaaattctgaaagaacatccagttgaaaacactcagctgcaaaatgaaaaattgtacctgtactacctgcagaatggaagagacatgtacgtggatcaggaattggatatcaatagactctcggactatgacgtagatcacattgtccctcagagcttcctcaaggatgattctatagataataaagtacttacgagatcggacaaaaatcgcggtaaatcggataacgtcccatcggaggaagtcgttaaaaagatgaaaaactattggcgtcaactgctgaacgccaagctgatcacacagcgtaagtttgataatctgactaaagccgaacgcggtggtcttagtgaactcgataaagcaggatttataaaacggcagttagtagaaacgcgccaaattacgaaacacgtggctcagatcctcgattctagaatgaatacaaagtacgatgaaaacgataaactgatccgtgaagtaaaagtcattaccttaaaatctaaacttgtgtccgatttccgcaaagattttcagttttacaaggtccgggaaatcaataactatcaccatgcacatgatgcatatttaaatgcggttgtaggcacggcccttattaagaaataccctaaactcgaaagtgagtttgtttatggggattataaagtgtatgacgttcgcaaaatgatcgcgaaatcagaacaggaaatcggtaaggctaccgctaaatactttttttattccaacattatgaatttttttaagaccgaaataactctcgcgaatggtgaaatccgtaaacggcctcttatagaaaccaatggtgaaacgggagaaatcgtttgggataaaggtcgtgactttgccaccgttcgtaaagtcctctcaatgccgcaagttaacattgtcaagaagacggaagttcaaacagggggattctccaaagaatctatcctgccgaagcgtaacagtgataaacttattgccagaaaaaaagattgggatccaaaaaaatacggaggctttgattcccctaccgtcgcgtatagtgtgctggtggttgctaaagtcgagaaagggaaaagcaagaaattgaaatcagttaaagaactgctgggtattacaattatggaaagatcgtcctttgagaaaaatccgatcgactttttagaggccaaggggtataaggaagtgaaaaaagatctcatcatcaaattaccgaagtatagtctttttgagctggaaaacggcagaaaaagaatgctggcctccgcgggcgagttacagaagggaaatgagctggcgctgccttccaaatatgttaattttctgtaccttgccagtcattatgagaaactgaagggcagccccgaagataacgaacagaaacaattattcgtggaacagcataagcactatttagatgaaattatagagcaaattagtgaattttctaagcgcgttatcctcgcggatgctaatttagacaaagtactgtcagcttataataaacatcgggataagccgattagagaacaggccgaaaatatcattcatttgtttaccttaaccaaccttggagcaccagctgccttcaaatatttcgataccacaattgatcgtaaacggtatacaagtacaaaagaagtcttggacgcaaccctcattcatcaatctattactggattatatgagacacgcattgatctttcacagctgggcggagacaagaagaaaaaactgaaactgcaccatcatcaccatcatcatcaccatcattgataactcgag

SEQ ID NO: 27 - DNA, 270 bp, Bacillus subtilis
atgaaatctaaatggatgtcaggtttgttgctcgttgcggtcgggttcagctttactcaggtgatggttctactttagatttacctacagtccaaacaacgagcaacgccagcccaagtcgaaatgagtccactaccaagatgcaggtgaaacagcaaacacagaagggaaaacatttcatattgcggcacgcaatcaaacatgatacgtccactttgtcgtttgtgtcttcccttttgtaaagtataacgccgtgcgttagtttgtact

SEQ ID NO: 28 - DNA, 23 bp, Bacillus subtilis
actcaggtgatggttcatgcagg

SEQ ID NO: 29 - DNA, 27 bp, Artificial Sequence
  primer
atgaaatctaaatggatgtcaggtttg

SEQ ID NO: 30 - DNA, 27 bp, Artificial Sequence
  primer
tcatgtttgattgcgtgccgcaatatg

SEQ ID NO: 31 - DNA, 4111 bp, Streptococcus pyogenes
atggacaagaagtactccatcggcctggacatcggcaccaactccgtcggctgggccgtcatcacggacgagtacaaggtcccgtcgaagaagttcaaggtgctgggcaacacggaccgccacagcatcaagaagaacctcatcggcgccctcctgttcgacagcggcgagacggccgaggcgacccgcctgaagcgcacggcgcggcggcggtacacgcgccgcaagaaccgcatctgctacctccaggagatcttctccaacgagatggcgaaggtcgacgactcgttcttccaccggctggaggagtccttcctggtcgaggaggacaagaagcacgagcggcaccccatcttcggcaacatcgtggacgaggtggcctaccacgagaagtacccgaccatctaccacctccggaagaagctggtggactcgaccgacaaggccgacctgcgcctgatctacctggccctcgcccacatgatcaagttccgcggccacttcctcatcgagggcgacctgaacccggacaactcggacgtcgacaagctgttcatccagctcgtgcagacctacaaccagctcttcgaggagaacccgatcaacgcctccggcgtggacgcgaaggccatcctgtcggcccgcctctcgaagtcccggcggctcgagaacctgatcgcccagctgccgggcgagaagaagaacggcctcttcggcaacctgatcgcgctctccctgggcctgacgccgaacttcaagtccaacttcgacctcgccgaggacgccaagctccagctgtccaaggacacgtacgacgacgacctggacaacctcctggcgcagatcggcgaccagtacgcggacctgttcctcgcggcgaagaacctctccgacgcgatcctgctcagcgacatcctccgcgtcaacaccgagatcaccaaggcgcccctgtccgcgagcatgatcaagcgctacgacgagcaccaccaggacctcaccctgctgaaggccctggtccgccagcagctcccggagaagtacaaggagatcttcttcgaccagtccaagaacggctacgcgggctacatcgacggcggcgcgtcccaggaggagttctacaagttcatcaagcccatcctggagaagatggacggcaccgaggagctgctggtcaagctgaaccgggaggacctcctgcgcaagcagcgcacgttcgacaacggctccatcccccaccagatccacctgggcgagctgcacgccatcctccggcgccaggaggacttctacccgttcctcaaggacaaccgcgagaagatcgagaagatcctcacgttccggatcccgtactacgtcggccccctcgcccgcggcaactcccgcttcgcgtggatgacgcgcaagtcggaggagacgatcaccccgtggaacttcgaggaggtggtggacaagggcgcctccgcgcagtcgttcatcgagcggatgaccaacttcgacaagaacctgccgaacgagaaggtgctgcccaagcacagcctcctgtacgagtacttcacggtctacaacgagctcaccaaggtgaagtacgtgacggagggcatgcggaagccggcgttcctgtccggcgagcagaagaaggccatcgtggacctgctgttcaagacgaaccggaaggtcaccgtcaagcagctgaaggaggactacttcaagaagatcgagtgcttcgactcggtcgagatcagcggcgtggaggaccgcttcaacgcgtcgctgggcacgtaccacgacctcctgaagatcatcaaggacaaggacttcctcgacaacgaggagaacgaggacatcctggaggacatcgtgctgaccctgacgctcttcgaggaccgggagatgatcgaggagcgcctcaagacctacgcgcacctgttcgacgacaaggtcatgaagcagctcaagcggcggcggtacaccggctggggccggctctcccgcaagctcatcaacggcatccgggacaagcagtcgggcaagaccatcctcgacttcctgaagtccgacggcttcgccaaccgcaacttcatgcagctgatccacgacgacagcctgacgttcaaggaggacatccagaaggcgcaggtgtcgggccagggcgactccctgcacgagcacatcgcgaacctggccggcagcccggccatcaagaagggcatcctgcagacggtcaaggtggtcgacgagctggtgaaggtgatgggccgccacaagcccgagaacatcgtcatcgagatggcccgggagaaccagaccacgcagaagggccagaagaacagccgcgagcgcatgaagcggatcgaggagggcatcaaggagctcggctcgcagatcctcaaggagcaccccgtcgagaacacccagctccagaacgagaagctctacctctactacctgcagaacggccgggacatgtacgtggaccaggagctcgacatcaaccggctgtcggactacgacgtcgaccacatcgtcccgcagtcgttcctgaaggacgacagcatcgacaacaaggtcctcacccgctcggacaagaaccggggcaagtccgacaacgtcccgtcggaggaggtcgtgaagaagatgaagaactactggcggcagctcctcaacgccaagctcatcacgcagcgcaagttcgacaacctgacgaaggcggagcggggcggcctcagcgagctcgacaaggcgggcttcatcaagcggcagctggtcgagacgcggcagatcacgaagcacgtggcccagatcctggactcgcgcatgaacaccaagtacgacgagaacgacaagctgatccgcgaggtcaaggtcatcaccctcaagtccaagctggtgagcgacttccgcaaggacttccagttctacaaggtgcgggagatcaacaactaccaccacgcccacgacgcgtacctgaacgcggtggtgggcacggcgctgatcaagaagtacccgaagctcgagagcgagttcgtctacggcgactacaaggtgtacgacgtgcggaagatgatcgcgaagagcgagcaggagatcggcaaggccacggcgaagtacttcttctacagcaacatcatgaacttcttcaagaccgagatcaccctggcgaacggcgagatccgcaagcgcccgctcatcgagaccaacggcgagacgggcgagatcgtgtgggacaagggccgcgacttcgcgaccgtccggaaggtgctgtccatgccccaggtcaacatcgtcaagaagacggaggtccagaccggcggcttcagcaaggagtccatcctgcccaagcggaacagcgacaagctgatcgcccggaagaaggactgggaccccaagaagtacggcggcttcgactcccccaccgtcgcctactcggtcctcgtggtcgccaaggtggagaagggcaagagcaagaagctgaagtccgtgaaggagctgctgggcatcaccatcatggagcggtcgtcgttcgagaagaaccccatcgacttcctggaggccaagggctacaaggaggtgaagaaggacctcatcatcaagctccccaagtactccctgttcgagctggagaacggccggaagcgcatgctcgccagcgcgggcgagctgcagaagggcaacgagctggcgctgccgagcaagtacgtcaacttcctgtacctcgcctcgcactacgagaagctgaagggctcgccggaggacaacgagcagaagcagctgttcgtggagcagcacaagcactacctcgacgagatcatcgagcagatctccgagttctccaagcgcgtcatcctggccgacgcgaacctggacaaggtcctgtccgcgtacaacaagcaccgcgacaagccgatccgggagcaggccgagaacatcatccacctgttcacgctgaccaacctcggcgcccccgcggccttcaagtacttcgacaccacgatcgaccggaagcggtacacgagcacgaaggaggtgctcgacgccaccctgatccaccagagcatcacgggcctgtacgagacccgcatcgacctctcccagctgggcggcgactgataga

SEQ ID NO: 32 - DNA, 20 bp, Artificial Sequence
  synthetic sequence
ttcatcgagtcctaccgtga

SEQ ID NO: 33 - DNA, 400 bp, Artificial Sequence
  synthetic sequence
cgacctgaccccggacacgctgccctactgggagctgcgggccacgtcctcgctggtcaccggaatcgagaaggcgttcatcgagtcctaccgcgtttaagctggactggggcctgtgcgacgggatgaccctcgacgcccggtgcaggagcgaccagtggccttagctcttccgcaagtagctcaggatggcgcaaattaccttaattaagtgacggctccttccagtacgtcctgatcgcggccgaccgcgtgtgagcgacgcggaccccggctgaacgactggtcccagcacgatgctggaattaattcactgccgaggaaggtcatgcaggactagcgccggctggcgcacactcgctgcgcctggggccgacttgctgaccagggtcgtgctacg

(19) United States
(12) Patent Application Publication
(10) Pub. No.: US 2018/0208945 A1
(43) Pub. Date: Jul. 26, 2018

(54) GENOME EDITING SYSTEMS AND METHODS OF USE

(71) Applicant: DANISCO US INC., Palo Alto, CA (US)

(72) Inventors: Kai BAO, Palo Alto, CA (US); Susan Mampusti MADRID, Millbrae, CA (US); Brian F. SCHMIDT, Paicines, CA (US)

(21) Appl. No.: 15/746,479

(22) PCT Filed: Jul. 28, 2016
(86) PCT No.: PCT/US2016/044489

§ 371 Date: Jan. 22, 2018

Related U.S. Application Data
(60) Provisional application No. 62/198,049, filed on Jul. 28, 2015.

(51) Int. Cl. C12N 15/90; C12N 9/22; C12N 15/11; C12N 15/80

(52) U.S. Cl. CPC C12N 15/902; C12N 9/22; C12N 2310/20; C12N 15/80; C12N 2800/80; C12N 15/11

(57) ABSTRACT

Compositions and methods are provided for genome editing at a target site in the genome of a filamentous fungal cell. The methods and compositions disclosed are drawn to a guide polynucleotide/Cas endonuclease system and donor polynucleotides with shorter homology arms (i.e., less than 500 bps) to a genomic locus of the fungal cell.

### CROSS-REFERENCE TO RELATED APPLICATIONS

The present application claims priority to U.S. Provisional Patent Application Ser. No. 62/198,049, filed on Jul. 28, 2015, which is hereby incorporated by reference in its entirety.

### SEQUENCE LISTING

The sequence listing text file submitted herewith via EFS contains the file “NB40972-WO-PCT_SEQ_LISTING.txt” created on Jul. 27, 2016, which is 22 kilobytes in size. This sequence listing complies with 37 C.F.R. § 1.52(e) and is incorporated herein by reference in its entirety.

### FIELD OF THE INVENTION

The present disclosure is generally related to the fields of molecular biology, genetics, biochemistry, genome editing and filamentous fungi. In certain embodiments, the present disclosure is directed to compositions and methods thereof for homologous recombination in a microbial cell and the microbial cells derived by such methods. In other embodiments, the present disclosure is directed to compositions and methods thereof for editing the genome of a microbial cell.

### BACKGROUND

It is known that inducing cleavage at a specific target site in genomic DNA can be used to introduce modifications at or near the target site. For example, homologous recombination for gene targeting has been shown to be enhanced when the targeted DNA site contains a double-strand break (see, e.g., Rudin et al., 1989; Smith et al.).

Given the site-specific nature of Cas systems, genome modification/engineering techniques based on these systems have been described, including in mammalian cells (see, e.g., Hsu et al., 2014). Cas-based genome engineering, when functioning as intended, confers the ability to target virtually any specific location within a complex genome, by designing a recombinant crRNA (or equivalently functional polynucleotide) in which the DNA-targeting region (i.e., the variable targeting domain) of the crRNA is homologous to a desired target site in the genome, and combining the crRNA with a Cas endonuclease (through any convenient and conventional means) into a functional complex in a host cell.

Although Cas-based genome engineering techniques have been applied to a number of different host cell types, even in filamentous fungal cells (see, e.g., Liu et al., 2015), these techniques have known limitations. For example, the efficiency of gene editing is strain-dependent, and generally multiple steps of molecular manipulation are required for donor DNA construction and the like.

Therefore, there remains a need for developing more effective, efficient or otherwise more robust or flexible Cas-based genome editing methods and compositions thereof for modifying/altering a genomic target site in a microbial cell.

### SUMMARY

The instant disclosure is generally directed to methods for editing the genome of a filamentous fungal cell. More particularly, in certain embodiments, the disclosure is directed to methods for genome editing in a filamentous fungal cell, comprising introducing into the filamentous fungal cell a Cas endonuclease, a guide polynucleotide, and a donor polynucleotide, wherein the donor polynucleotide comprises at least one homology arm, wherein the homology arm is less than 500 nucleotides in length and comprises sequence homology to a targeted genomic locus of the fungal cell, wherein the Cas endonuclease and guide polynucleotide form a complex that enables the Cas endonuclease to act at or near the targeted genomic locus of the fungal cell. In certain embodiments, the donor polynucleotide is inserted (incorporated) into the targeted genomic locus of the fungal cell.

In certain other embodiments, the donor polynucleotide further comprises a nucleotide sequence of interest which is either upstream (5′) and operably linked to the homology arm or downstream (3′) and operably linked to the homology arm. The nucleotide sequence of interest can comprise a single nucleotide, two nucleotides, three nucleotides, etc. In other embodiments, a nucleotide sequence of interest is a polynucleotide generally comprising five (5) or more nucleotides. In certain embodiments, the homology arm is less than 350 nucleotides in length. In other embodiments, the homology arm is less than 150 nucleotides in length. In certain other embodiments, the homology arm is between 100-40 nucleotides in length.

In particular embodiments, the nucleotide sequence of interest is inserted (incorporated) into the targeted genomic locus of the fungal cell. In other embodiments, the inserted donor nucleic acid or polynucleotide results in a genome modification selected from the group consisting of a DNA deletion, a DNA disruption, a DNA insertion, a DNA inversion, a DNA point mutation, a DNA replacement, a DNA knock-in, a DNA knock-out and a DNA knock-down.

In yet other embodiments, the donor polynucleotide comprises a homology arm upstream (5′) and operably linked to a nucleotide sequence of interest and a homology arm downstream (3′) and operably linked to the same nucleotide sequence of interest, wherein at least one of the two homology arms are less than 500 nucleotides in length. In particular embodiments, the nucleotide sequence of interest is inserted into the targeted genomic locus of the fungal cell. In certain embodiments, at least one homology arm is less than 350 nucleotides in length. In certain other embodiments, at least one homology arm is less than 150 nucleotides in length. In another embodiment, at least one homology arm is between 100-40 nucleotides in length. In other embodiments, both homology arms are less than 500 nucleotides. In certain other embodiments, the nucleotide sequence of interest comprises at least one heterologous nucleotide. In yet other embodiments, the nucleotide sequence of interest comprises a heterologous polynucleotide sequence.

In other embodiments, the Cas endonuclease is a Cas nickase or a functional variant thereof. In particular embodiments, the Cas endonuclease is a Cas9 endonuclease or a functional variant thereof. In other embodiments, the Cas9 endonuclease is a Cas9 endonuclease derived from a genus selected from the group consisting of Streptococcus sp., Campylobacter sp., Neisseria sp., Francisella sp. and Pasteurella sp.

In other embodiments, the introducing step comprises introducing a polynucleotide construct comprising an expression cassette for expressing the Cas endonuclease (or a functional variant thereof) in the fungal cell. In another embodiment, the introducing step comprises introducing a polynucleotide construct comprising an expression cassette for expressing the guide polynucleotide in the fungal cell. In another embodiment, the introducing step comprises introducing into the fungal cell a circular polynucleotide construct comprising an expression cassette for the Cas endonuclease, an expression cassette for the guide RNA, and the donor DNA. In another embodiment, the introducing step comprises directly introducing the guide polynucleotide or Cas endonuclease into the fungal cell.

In certain embodiment, the Cas endonuclease (or a functional variant thereof) is operably linked to a nuclear localization signal.

In other embodiments, the donor polynucleotide is a double strand DNA. In certain other embodiment, the donor polynucleotide is a single strand DNA.

In other embodiments, the filamentous fungal cell is selected from the genus consisting of Trichoderma, Penicillium, Aspergillus, Humicola, Chrysosporium, Fusarium, Myceliophthora, Neurospora and Emericella.

Other embodiments of the disclosure are directed to recombinant filamentous fungal cells produced by the method and compositions disclosed herein.

### BRIEF DESCRIPTION OF THE DRAWINGS

FIGS. 1A-1C: depict a 100-mer single strand donor template with a 19 nucleotide insertion sequence into the pyr4 locus (TS2). FIG. 1A: The schematic of pyr4 genomic locus target site 2 (TS2). 1F & 1R, MH179 & 180 are the PCR primers used for analysis. The single strand oligonucleotide of 100 bases is the donor template. FIG. 1B: The 100-nt upper strand of single strand donor DNA. The 19-nt insertion sequence will create 2 restriction sites (Pme1, Pac1) and the stop codon (TAA) in 3 different readings frames creating a loss of function mutation in the pyr4 gene locus. FIG. 1C: The donor templates for homologous recombination: 100 bases single strand oligonucleotides: in 1C-1, the 100-nt upper strand, and in 1C-2, the 100-nt lower strand sequences (complementary strand to target site).

FIGS. 2A-2C: depict homology directed repair using the 100 bases single strand DNA template and efficiency of genome editing. FIG. 2A: PCR amplifications were carried out on DNA extracted from FOA resistant colonies using primers 1F & 1R, SEQ ID NO: 12 & 13. PCR amplifications across the target site TS2 in the pyr4 gene resulting in 1.2 kb product. FIG. 2B: Restriction digestions of PCR products showed the presence of Pac1. FIG. 2C: Restriction digestions of PCR products showed the presence of Pme1.

FIGS. 3A-3C: depict homology directed repair using the 200 bases single strand DNA template and efficiency of genome editing. FIG. 3A: Sequence of single strand DNA template of 200 bases. FIG. 3B: PCR amplifications were carried out on DNA extracted from FOA resistant colonies using primers 1F & 1R, SEQ ID NO: 12 & 13. PCR amplifications across the target site TS2 in the pyr4 gene resulting in 1.2 kb product. FIG. 3C: Restriction digestions of PCR products showed the presence of Pac1.

FIG. 4: depicts sequence alignment of wild type pyr4 gene and the pyr4 genes from FOA resistant strains indicating Cas9 mediated Homology Directed Repair using single strand DNA template of 100 (A) and 200 (B) bases.

FIG. 5: depicts the sequences of double strand DNA template with the insertion codons and the flanking homologous arms.

FIGS. 6A-6C: depict double strand DNA donor repair template of length 730 bps. FIG. 6A: Schematic diagram showing the 730 bps double strand DNA. FIG. 6B: PCR amplification of the pyr4 gene locus across TS2 using primers (SEQ ID NOs:14 & 15) from the genomic DNA of FOA resistant colonies. FIG. 6C: Restriction enzyme digestion of the PCR products by using PacI.

FIGS. 7A-7C: depict double strand DNA donor repair template of length 1100 bps. FIG. 7A: Schematic diagram showing the 1100 bps ds DNA. FIG. 7B: PCR amplification of the pyr4 gene locus across TS2 using primers (SEQ ID NOs:14 & 15) from the genomic DNA of FOA resistant colonies. FIG. 7C: Restriction enzyme digestion of the PCR products by using PacI.

FIG. 8: depicts the pSB-SpyCas9 expression vector.

FIGS. 9A-9B: depict the SDS-PAGE analysis of intracellularly expressed Cas9 in Bacillus subtilis showing high levels of production of Cas9. FIG. 9A depicts the Western Blot of the SDS-PAGE. FIG. 9B depicts the Coomassie stained SDS-PAGE, as per Example 8.

FIG. 10 depicts an expression cassette as per Example 10, which shows the 2 kb homology arms, the Cas9 gene, and the guide RNA in a single plasmid, used for Cas9-mediated targeted disruption of the Streptomyces MIB gene.

FIG. 11 depicts an expression cassette showing the Cas9 gene, the guide RNAs in a single plasmid, but without the 2 kb homology arms, for targeted disruption of the MIB gene. The lack of the homology arms allowed the use of ultramers as donor for homologous recombination as per descriptions of Example 10.

### DETAILED DESCRIPTION

### Overview

In certain embodiments, the present disclosure relates to methods and compositions thereof for efficient homologous recombination in a microbial cell. In certain other embodiments, the present disclosure is directed to methods and compositions thereof for genome editing in a microbial cell. In other embodiments, the present disclosure relates to microbial cells made by such methods and compositions of the present disclosure.

### Abbreviations and Acronyms

The following abbreviations/acronyms have the following meanings unless otherwise specified:

cDNA complementary DNA

DNA deoxyribonucleic acid

EDTA ethylenediaminetetraacetic acid

kDa kiloDalton

MW molecular weight

PEG polyethyleneglycol

ppm parts per million, e.g., μg protein per gram dry solid

RNA ribonucleic acid

SDS-PAGE sodium dodecyl sulfate polyacrylamide gel electrophoresis

sp. species

Tm melting temperature

w/v weight/volume

w/w weight/weight

° C. degrees Centigrade

H₂O water

g or gm grams

μg micrograms

mg milligrams

kg kilograms

μL and μl microliters

mL and ml milliliters

mm millimeters

μm micrometer

M molar

mM millimolar

μM micromolar

U units

sec seconds

min(s) minute/minutes

hr(s) hour/hours

Tris-HCl tris(hydroxymethyl)aminomethane hydrochloride

HEPES 4-(2-hydroxyethyl)-1-piperazineethanesulfonic acid

CV column volumes

### Definitions

Prior to describing the present compositions and methods, the following terms and phrases are defined. Terms not defined herein should be accorded their ordinary meaning as is known and used in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior disclosure. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to “exclude” any optional element. As such, this statement is intended to serve as antecedent basis for use of such exclusive terminology as “solely”, “only”, “not including”, “excluding” and the like in connection with the recitation of claim elements, or use of a “negative” limitation.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising”, as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, a polypeptide referred to as a “Cas endonuclease” or having “Cas endonuclease activity” relates to a CRISPR associated (Cas) polypeptide encoded by a Cas gene, wherein the Cas polypeptide is capable of cutting a target DNA sequence when functionally coupled with one or more guide polynucleotides (see, e.g., U.S. Pat. No. 8,697,359). Variants of Cas endonucleases that retain guide polynucleotide directed endonuclease activity are also included in this definition. The Cas endonucleases employed in the donor DNA insertion methods detailed herein are endonucleases that introduce double-strand breaks into the DNA at the target site. A Cas endonuclease is guided by the guide polynucleotide to recognize and cleave a specific target site in double stranded DNA (e.g., at a target site in the genome of a cell).

As used herein, the term “genome-editing” is a type of genetic engineering in which DNA is inserted, replaced, or removed from a genome using artificially engineered nucleases, or “molecular scissors.” It is a useful tool to elucidate the function and effect of a gene or protein in a sequence specific manner.

As used herein, the term “guide polynucleotide” relates to a polynucleotide sequence that can form a complex with a Cas endonuclease and enables the Cas endonuclease to recognize and cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited to, Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. In one embodiment, the size of the fragment of the crRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides). In certain embodiments, the RNA that guides the RNA/Cas9 endonuclease complex is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In one embodiment of the disclosure, the single guide RNA comprises a crRNA or crRNA fragment and a tracrRNA or tracrRNA fragment of the type II CRISPR/Cas system that can form a complex with a type II Cas endonuclease, wherein the guide RNA/Cas endonuclease complex can direct the Cas endonuclease to a fungal cell genomic target site, enabling the Cas endonuclease to introduce a double strand break into the genomic target site.

One aspect of using a single guide polynucleotide versus a duplex guide polynucleotide is that only one expression cassette needs to be made to express the single guide polynucleotide in a target cell.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetra-loop sequence, such as, but not limited to, a GAAA tetra-loop sequence.

Nucleotide sequence modification of the guide polynucleotide, and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

As used herein, the term “guide polynucleotide/Cas endonuclease system” (and equivalents) includes a complex of a Cas endonuclease and a guide polynucleotide (single or double) that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is appropriately oriented at the 3′ end of the target sequence.

As used herein, the terms “functional fragment”, “fragment that is functionally equivalent”, “functionally equivalent fragment”, and the like, are used interchangeably and refer to a portion or subsequence of a parent polypeptide that retains the qualitative enzymatic activity of the parent polypeptide. For example, a functional fragment of a Cas endonuclease retains the ability to create a double-strand break with a guide polynucleotide. It is noted here that a functional fragment may have altered quantitative enzymatic activity as compared to the parent polypeptide.

Relatedly, the terms “functional variant”, “variant that is functionally equivalent”, “functionally equivalent variant”, and the like are used interchangeably and refer to a variant of a parent polypeptide that retains the qualitative enzymatic activity of the parent polypeptide. For example, a functional variant of a Cas endonuclease retains the ability to create a double-strand break with a guide polynucleotide. It is noted here that a functional variant may have altered quantitative enzymatic activity as compared to the parent polypeptide.

Fragments and variants can be obtained via any convenient method, including site-directed mutagenesis and synthetic construction.

As used herein, the term “codon-modified gene” or “codon-preferred gene” or “codon-optimized gene” is a gene having its frequency of codon usage designed to mimic the frequency of preferred codon usage of the host cell. The nucleic acid changes made to codon-optimize a gene are “synonymous”, meaning that they do not alter the amino acid sequence of the encoded polypeptide of the parent gene. However, both native and variant genes can be codon-optimized for a particular host cell, and as such no limitation in this regard is intended.

As used herein, the term “coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, 5′ untranslated sequences, 3′ untranslated sequences, introns, polyadenylation target sequences, RNA processing sites, effector binding sites, and stem-loop structures.

As used herein, the term “promoter” refers to a DNA sequence capable of controlling the expression of a coding sequence or functional RNA. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. As is well-known in the art, promoters can be categorized according to their strength and/or the conditions under which they are active, e.g., constitutive promoters, strong promoters, weak promoters, inducible/repressible promoters, tissue-specific/developmentally regulated promoters, cell-cycle dependent promoters, etc.

As used herein, the term “RNA transcript” refers to the product resulting from RNA polymerase-catalyzed transcription of a DNA sequence. “Messenger RNA” or “mRNA” refers to the RNA that is without introns and that can be translated into protein by the cell. “cDNA” refers to a DNA that is complementary to, and synthesized from, a mRNA template using the enzyme reverse transcriptase. “Sense” RNA refers to RNA transcript that includes the mRNA and can be translated into protein within a cell or in vitro. “Antisense RNA” refers to an RNA transcript that is complementary to all or part of a target primary transcript or mRNA, and that, under certain conditions, blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated into a polypeptide but yet has an effect on cellular processes. The terms “complement” and “reverse complement” are used interchangeably herein with respect to mRNA transcripts, and are meant to define the antisense RNA of the message.

As used herein, the term “functionally attached” or “operably linked” means that a regulatory region or functional domain of a polypeptide or polynucleotide sequence having a known or desired activity, such as a promoter, enhancer region, terminator, signal sequence, epitope tag, etc., is attached to or linked to a target (e.g., a gene or polypeptide) in such a manner as to allow the regulatory region or functional domain to control the expression, secretion or function of that target according to its known or desired activity. For example, a promoter is operably linked with a coding sequence when it is capable of regulating the expression of that coding sequence (i.e., the coding sequence is under the transcriptional control of the promoter).

As used herein, the term “PCR” or “polymerase chain reaction” is a technique for the synthesis of specific DNA segments and consists of a series of repetitive denaturation, annealing, and extension cycles and is well known in the art.

As used herein, the term “recombinant,” when used in reference to a biological component or composition (e.g., a cell, nucleic acid, polypeptide/enzyme, vector, etc.) indicates that the biological component or composition is in a state that is not found in nature. In other words, the biological component or composition has been modified by human intervention from its natural state. For example, a recombinant cell encompasses a cell that expresses one or more genes that are not found in its native parent (i.e., non-recombinant) cell, a cell that expresses one or more native genes in an amount that is different than its native parent cell, and/or a cell that expresses one or more native genes under different conditions than its native parent cell. Recombinant nucleic acids may differ from a native sequence by one or more nucleotides, be operably linked to heterologous sequences (e.g., a heterologous promoter, a sequence encoding a non-native or variant signal sequence, etc.), be devoid of intronic sequences, and/or be in an isolated form. Recombinant polypeptides/enzymes may differ from a native sequence by one or more amino acids, may be fused with heterologous sequences, may be truncated or have internal deletions of amino acids, may be expressed in a manner not found in a native cell (e.g., from a recombinant cell that over-expresses the polypeptide due to the presence in the cell of an expression vector encoding the polypeptide), and/or be in an isolated form. It is emphasized that in some embodiments, a recombinant polynucleotide or polypeptide/enzyme has a sequence that is identical to its wild-type counterpart but is in a non-native form (e.g., in an isolated or enriched form).

As used herein, the terms “plasmid”, “vector” and “cassette” refer to an extra chromosomal element that carries a polynucleotide sequence of interest, e.g., a gene of interest to be expressed in a cell (an “expression vector” or “expression cassette”). Such elements are generally in the form of double-stranded DNA and may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. The polynucleotide sequence of interest may be a gene encoding a polypeptide or functional RNA that is to be expressed in the target cell. Expression cassettes/vectors generally contain a gene with operably linked elements that allow for expression of that gene in a host cell.

As used herein, the term “expression”, as used herein, refers to the production of a functional end-product (e.g., an mRNA, guide RNA, or a protein) in either precursor or mature form.

As used herein, the term “introduced” in the context of inserting a polynucleotide or polypeptide into a cell (e.g., a recombinant DNA construct/expression construct) refers to any method for performing such a task, and includes any means of “transfection”, “transformation”, “transduction”, physical means, or the like, to achieve introduction of the desired biomolecule.

By “introduced transiently”, “transiently introduced”, “transient introduction”, “transiently express” and the like is meant that a biomolecule is introduced into a host cell (or a population of host cells) in a non-permanent manner. With respect to double stranded DNA, transient introduction includes situations in which the introduced DNA does not integrate into the chromosome of the host cell and thus is not transmitted to all daughter cells during growth as well as situations in which an introduced DNA molecule that may have integrated into the chromosome is removed at a desired time using any convenient method (e.g., employing a cre-lox system, by removing positive selective pressure for an episomal DNA construct, by promoting looping out of all or part of the integrated polynucleotide from the chromosome using a selection media, etc.). No limitation in this regard is intended. In general, introduction of RNA (e.g., a guide RNA, a messenger RNA, ribozyme, etc.) or a polypeptide (e.g., a Cas polypeptide) into host cells is considered transient in that these biomolecules are not replicated and indefinitely passed down to daughter cells during cell growth. With respect to the Cas/guide RNA complex, transient introduction covers situations when either of the components is introduced transiently, as both biomolecules are needed to exert targeted Cas endonuclease activity. Thus, transient introduction of a Cas/guide RNA complexes includes embodiments where either one or both of the Cas endonuclease and the guide RNA are introduced transiently. For example, a host cell having a genome-integrated expression cassette for the Cas endonuclease (and thus not transiently introduced) into which a guide RNA is transiently introduced can be said to have a transiently introduced Cas/guide RNA complex (or system) because the functional complex is present in the host cell in a transient manner.

As used herein, the term “mature” protein refers to a post-translationally processed polypeptide (i.e., one from which any pre- or pro-peptides present in the primary translation product have been removed). “Precursor” protein refers to the primary product of translation of mRNA (i.e., with pre- and pro-peptides still present). Pre- and pro-peptides may be, but are not limited to, intracellular localization signals.

As used herein, the term “fungal cell”, “fungi”, “fungal host cell”, and the like, as used herein includes the phyla Ascomycota, Basidiomycota, Chytridiomycota, and Zygomycota (as defined by Hawksworth et al., 1995), as well as the Oomycota (Hawksworth et al., 1995) and all mitosporic fungi (Hawksworth et al., 1995). In certain embodiments, the fungal host cell is a yeast cell, wherein the term “yeast” is meant ascosporogenous yeast (Endomycetales), basidiosporogenous yeast, and yeast belonging to the Fungi Imperfecti (Blastomycetes). As such, a yeast host cell includes a Candida, Hansenula, Kluyveromyces, Pichia, Saccharomyces, Schizosaccharomyces, or Yarrowia cell. Species of yeast include, but are not limited to, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Kluyveromyces lactis, and Yarrowia lipolytica.

As used herein, the term “filamentous fungal cell” includes all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma Trichoderma longibrachiatum, Trichoderma reesei, and Trichoderma viride.

As used herein, the terms “target site”, “target sequence”, “genomic target site”, “genomic target sequence” (and equivalents thereof) are used interchangeably herein and refer to a polynucleotide sequence in the genome of a fungal cell at which a Cas endonuclease cleavage is desired to promote a genome modification, e.g., insertion of a donor DNA and subsequent deletion of a genomic region of interest. The context in which this term is used, however, can slightly alter its meaning. For example, the target site for a Cas endonuclease is generally very specific and can often be defined to the exact nucleotide position, whereas in some cases the target site for a desired genome modification can be defined more broadly than merely the site at which DNA cleavage occurs, e.g., a genomic locus or region that is to be deleted from the genome. Thus, in certain cases, the genome modification that occurs via the activity of Cas/guide RNA DNA cleavage is described as occurring “at or near” the target site. The target site can be an endogenous site in the fungal cell genome, or alternatively, the target site can be heterologous to the fungal cell and thereby not be naturally occurring in the genome, or the target site can be found in a heterologous genomic location compared to where it occurs in nature.

As used herein, the term “nucleic acid” means a polynucleotide and includes a single or a double-stranded polymer of deoxyribonucleotide or ribonucleotide bases. Nucleic acids may also include fragments and/or modified nucleotides. Thus, the terms “polynucleotide”, “nucleic acid sequence”, “nucleotide sequence” and “nucleic acid fragment” are used interchangeably to denote a polymer of RNA and/or DNA that is single-stranded or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

As used herein, the term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “substantially similar” or “substantially identical,” in the context of at least two nucleic acids or polypeptides, means that a polynucleotide or polypeptide comprises a sequence that has at least 90%, at least 91%, at least 92%, at least 93%, at least 94%, at least 95%, at least 96%, at least 97%, at least 98%, or even at least 99% identical to a parent or reference sequence, or does not include amino acid substitutions, insertions, deletions, or modifications made only to circumvent the present description without adding functionality.

As used herein, the term “sequence identity” or “identity” in the context of nucleic acid or polypeptide sequences refers to the nucleic acid bases or amino acid residues in two sequences that are the same when aligned for maximum correspondence over a specified comparison window.

As used herein, the term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

As used herein, the term “Clustal V method of alignment” corresponds to the alignment method labeled Clustal V (Higgins and Sharp, 1989; Higgins et al., 1992) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

As used herein, the term “Clustal W method of alignment” corresponds to the alignment method labeled Clustal W (Higgins and Sharp, 1989; Higgins et al., 1992) and found in the MegAlign™ v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, 1989). GAP uses the algorithm of Needleman and Wunsch, (1970), to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

As used herein, the term “gene” includes a nucleic acid fragment that encodes and is capable to express a functional molecule such as, but not limited to, a specific polypeptide (e.g., an enzyme) or a functional RNA molecule (e.g., a guide RNA, an anti-sense RNA, ribozyme, etc.), and includes regulatory sequences preceding (5′ non-coding sequences) and/or following (3′ non-coding sequences) the coding sequence. “Native gene” refers to a gene as found in nature with its own regulatory sequences.

As used herein, the term “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, a mutated gene comprises an alteration that results from a guide polynucleotide/Cas endonuclease system as disclosed herein. A mutated fungal cell is a fungal cell comprising a mutated gene.

As used herein, the term “targeted mutation” is a mutation in a native gene that was made by altering a target sequence within the native gene using a method involving a double-strand-break-inducing agent that is capable of inducing a double-strand break in the DNA of the target sequence as disclosed herein or known to one skilled in the art.

The term “polynucleotide modification template” refers to a polynucleotide that comprises at least one nucleotide modification when compared to the nucleotide sequence to be edited. A nucleotide modification can include, for example: (i) a replacement of at least one nucleotide, (ii) a deletion of at least one nucleotide, (iii) an insertion of at least one nucleotide, or (iv) any combination of (i)-(iii). Optionally, the polynucleotide modification template can further comprise homologous nucleotide sequences flanking the at least one nucleotide modification, wherein the flanking homologous nucleotide sequences provide sufficient homology to the desired nucleotide sequence to be edited. The flanking homologous sequences are alternatively referred to herein as “homology arms”.

As used herein, the terms “donor DNA”, “donor nucleic acid sequence” and “donor polynucleotide” refer to a polynucleotide modification template comprising a “polynucleotide of interest” to be inserted into the target site of the Cas endonuclease (i.e., in conjunction with the activity of a Cas endonuclease/guide polynucleotide complex). In certain embodiments, the donor DNA construct comprises at least one region of homology (a “homology arm”) that flanks the polynucleotide of interest (i.e., the homology arm is upstream (5′) or downstream (3′) of the polynucleotide of interest). Thus, in certain embodiments, a donor DNA construct comprising at least one homology arm shares homology to a genomic region present in or flanking the (Cas) target site of the fungal cell genome. In other embodiments, a donor DNA construct comprises both an upstream (5′) homology arm and a downstream (3′) homology arm flanking the polynucleotide of interest. Thus, in other embodiments of the disclosure, a donor DNA construct comprises a first homology arm which is upstream (5′) and operably linked to the polynucleotide of interest and a second homology arm which is downstream (3′) and operably linked to the polynucleotide of interest. The first homology arm and second homology arm of the donor DNA construct share homology to a first genomic region and a second genomic region of the (Cas) target site of the fungal cell genome, respectively. Thus, in particular embodiments, a donor DNA (or polynucleotide modification template) comprises two homologous sequences (i.e., 5′ and 3′ homology arms) separated by a polynucleotide sequence of interest (or a base pair of interest) that is heterologous to the sequence at the target site. Homologous recombination between the genomic target site and the two donor DNA homology arms typically results in the editing of the sequence at the target site.

By “homologous” is meant DNA sequences that are similar. For example, a “region homologous to a genomic sequence” that is found on the donor DNA is a region of DNA that has a similar sequence to a given “genomic sequence” in the fungal cell genome. Collectively, the sequence homologous to a genomic sequence in the genomic locus and the genomic sequence itself are sometimes referred to herein as “the repeat sequences”. A homologous region can be of any length that is sufficient to promote looping-out of the loop-out target region via homologous recombination between the repeat sequence and the homologous genomic sequence (which can be selected for under selective culture conditions).

For example, the repeat sequence (homology arm) can comprise at least 50-55, 50-60, 50-65, 50-70, 50-75, 50-80, 50-85, 50-90, 50-95, 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, 50-2000, 50-2100, 50-2200, 50-2300, 50-2400, 50-2500, 50-2600, 50-2700, 50-2800, 50-2900, 50-3000, 50-3100 or more bases in length. “Sufficient homology” indicates that two polynucleotide sequences (e.g., direct repeat sequences in the donor DNA and the genome of fungal cell) have sufficient structural similarity to loop-out the sequence in between the repeat sequences, e.g., under appropriate selective culture conditions. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of at least one of the sequences.

As used herein, the term “genomic region” or “genomic locus” is a segment of a chromosome in the genome of a fungal cell that is present on either side of the target site (e.g., including the genomic deletion target and the genomic repeat sequence that is homologous to the repeat sequence in a donor DNA) or, alternatively, also comprises a portion of the target site. The genomic region can comprise at least 50-55, 50-60, 50-65, 50-70, 50-75, 50-80, 50-85, 50-90, 50-95, 50-100, 50-200, 50-300, 50-400, 50-500, 50-600, 50-700, 50-800, 50-900, 50-1000, 50-1100, 50-1200, 50-1300, 50-1400, 50-1500, 50-1600, 50-1700, 50-1800, 50-1900, 50-2000, 50-2100, 50-2200, 50-2300, 50-2400, 50-2500, 50-2600, 50-2700, 50-2800, 50-2900, 50-3000, 50-3100 or more bases.

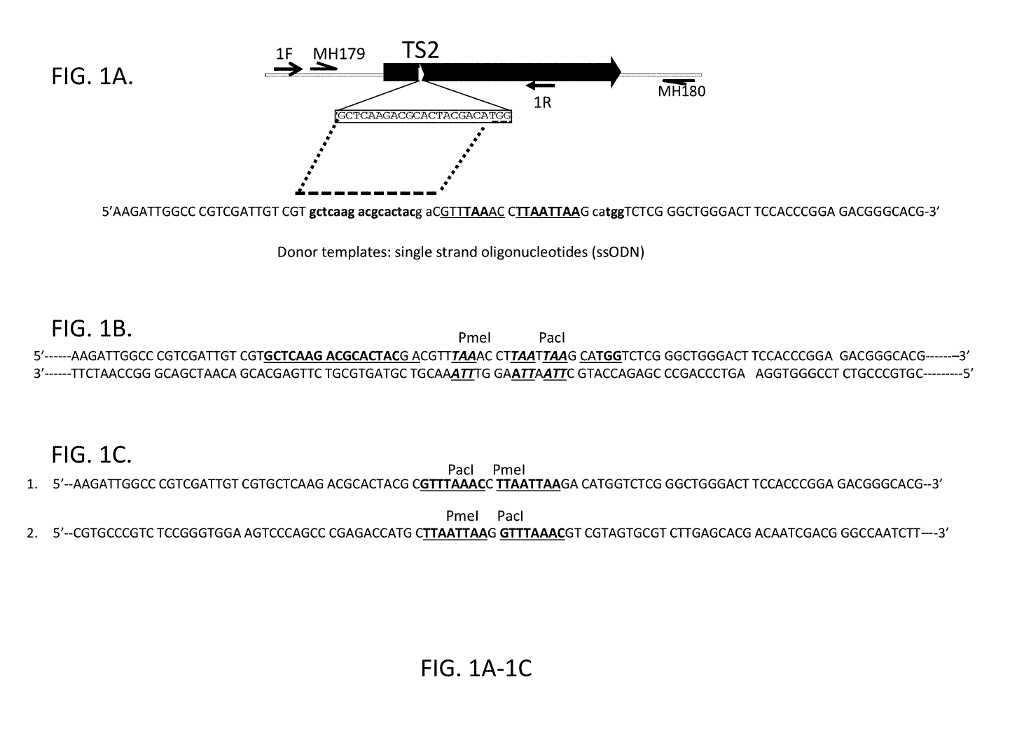

As used herein, the term “genomic deletion target” and equivalents is the sequence in the fungal genome that a user wants to delete according to aspects of the present disclosure (e.g., see FIG. 1). A “loop-out target region” and equivalents is the region between direct repeats (e.g., the genomic repeat sequence and the repeat sequence in the donor DNA that is homologous to the genomic repeat sequence) that is looped-out by homologous recombination between the direct repeats in the fungal genome. In certain embodiments, the loop-out target region includes the genomic deletion target and the selectable marker on the donor DNA inserted at the target site in the fugal genome. A phenotypic marker is a screenable or selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds and antibiotics, such as, chlorimuron ethyl, benomyl, Basta, and hygromycin phosphotransferase (HPT); DNA segments that encode products which are otherwise lacking in the recipient cell (e.g., tRNA genes, auxotrophic markers, dominant heterologous marker-amdS); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc. and, the inclusion of a DNA sequences required for a specific modification (e.g., methylation) that allows its identification.

As used herein, the term “signal sequence” is a sequence of amino acids attached to the N-terminal portion of a protein, which facilitates the secretion of the protein outside the cell. The mature form of an extracellular protein lacks the signal sequence, which is cleaved off during the secretion process.

As used herein, the terms “polypeptide” and “protein” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “heterologous” nucleic acid construct or sequence has a portion of the sequence which is not native or existing in a native form to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present in the native state, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “host cell”, includes any fungus, whether a unicellular organism, a cell derived from a multicellular organism and placed in tissue culture, or a cell present as part of a multicellular organism, which is susceptible to transformation with a nucleic acid construct according to the disclosure. Such host cells, such as yeast and other fungal cells, or bacteria may be used for replicating DNA and producing polypeptides encoded by nucleotide sequences as used in the disclosure. Suitable cells for the present invention are generally filamentous fungi or yeasts. Particularly preferred are cells from filamentous fungi, preferably Aspergillus, such as A. niger and A. tubingensis. Other preferred organisms include any one of Aspergillus oryzae, A. awamori, Trichoderma reesei, Trichoderma viride and Trichoderma longibrachiatum.

As used herein, the term “introduced” in the context of inserting a nucleic acid sequence into a cell, means “transfection”, “transformation” or “transduction,” as known in the art.

As used herein, “transformed” means a cell has been transformed by use of recombinant DNA techniques. Transformation typically occurs by insertion of one or more nucleotide sequences into a cell. The inserted nucleotide sequence may be a heterologous nucleotide sequence, i.e., is a sequence that is not natural to the cell that is to be transformed, such as a fusion protein.

As used herein, the term “expression” refers to the process by which a polypeptide is produced based on a nucleic acid sequence. The process includes both transcription and translation.

### Methods and Compositions for Modifying a Microbial Cell Genome

The present disclosure relates to methods for homologous recombination in a microbial cell and the microbial cells made by such methods. The present disclosure also pertains to methods for genome editing in a microbial cell.

### 1. Methods of Gene Editing

In certain embodiments, fungi of the disclosure are biotechnologically applied microbes used for the production of proteins including different hydrolytic enzymes such as cellulases and xylanases. For example, Hypocrea jecorina (synonym Trichoderma reesei) is arguably the best studied cellulolytic fungus, and its cellulases and hemicellulases are currently at the forefront of investigation for the enzymatic conversion of renewable lignocellulosic biomass to biofuels. Therefore, there is a need to develop efficient molecular tools to further improve industrial protein/cellulase production and to obtain new insights regarding the mechanism for cellulase or hemicellulase gene regulation.

Gene targeting by homologous recombination (HR) is a key technique to study the function of genes and to alter the characteristics of fungal strains for different applications. Nevertheless, in contrast to the yeast Saccharomyces cerevisiae which has a high rate of homologous gene (HR) targeting, the non-homologous end joining pathway (NHEJ) (Bleuyard et al., 2006) pathway seems to be the dominant mode of DNA integration in other fungi, including various filamentous fungi, leading to direct ligation of strands without sequence homology.

HR for gene targeting has been shown to be enhanced when the targeted DNA site contains a double-strand break (see, e.g., Rudin et al., 1989; Smith et al.). The type II clustered regularly interspaced short palindromic repeats (CRISPR)/CRISPR-associated gene (Cas) system, the most popular genome-editing tool at this time, can catalyze a double-strand break (DSB) in the target DNA composed of a 20-bp sequence matching the protospacer of the guide RNA (gRNA) and an adjacent downstream 5′-NGG nucleotide sequence (termed as the protospacer-adjacent motif (PAM)) (see, e.g., Cong et al., 2013).

Over the past 2 years, many studies have demonstrated that the CRISPR/Cas9 system is a powerful genome editing method that facilitates genetic alterations in genomes in a variety of organisms. Although Cas-based genome engineering technologies have been applied to a number of different host cell types, even in filamentous fungal cells (Liu et al., 2015), they have limitations including, for example, the gene editing is cas9 expressed microbial strain dependent, and multiple steps of molecular manipulation are needed for donor DNA construction, etc.

Thus, based on the foregoing, there remains a need in the art for developing more robustly effective and efficient Cas-based genome editing methods and compositions thereof for modifying/altering a genomic target site in a microbial cell.

2. Short Homology Arms in Donor DNA required for Gene Editing

Methods are provided herein employing a guide RNA/Cas endonuclease system for inserting a donor DNA with one or more short homology arms at a target site in the genome of a microbial cell (e.g., a filamentous fungal cell).

In a first aspect, the present disclosure provides improved methods for targeted gene editing in the genomes of microbial cells, (e.g., filamentous fungal cells), via homologous recombination of donor DNAs with targeted genomic loci in such microbial cells. Such a method comprises: (a) introducing into a population of microbial cells a Cas endonuclease, a guide RNA, and a donor DNA comprising a domain with homology to a genomic locus of the microbial cell, wherein the length of one or both of the homology arms in the donor DNA are short, ranging from 40 bps to 500 bps, such as, e.g., from 40 bps to 450 bps, or from 45 bps to 400 bps, or 50 bps to 350 bps, or 55 bps to 300 bps, and so on, wherein the Cas endonuclease and guide RNA are capable of forming a complex that enables the Cas endonuclease to act at a target site, in or near the genomic locus of the microbial cells and (b) identifying at least one microbial cell from the population of microbial cells in which homologous recombination of the donor DNA with the genomic locus has occurred; wherein the Cas endonuclease, the guide RNA, or both are introduced into the population of microbial cells.

In a second aspect, the disclosure provides a method of genome editing in a microbial cell, the method comprising: (a) introducing into a population of microbial cells a Cas endonuclease, a guide RNA, and a donor DNA comprising a domain with homology to a genomic locus of the microbial cell, wherein the length of at least one of the homology arms in the donor DNA is short, ranging from 40 bps to 500 bps, such as, e.g., from 45 bps to 450 bps, from 50 bps to 400 bps, from 55 bps to 350 bps, from 55 bps to 300 bps, and so on, wherein the Cas endonuclease and guide RNA are capable of forming a complex that enables the Cas endonuclease to act at a target site, in or near a genomic locus of the genome of the microbial cells; and (b) identifying at least one microbial cell from the population of microbial cells in which DNA modification at the target site in the genomic locus has occurred, wherein the Cas endonuclease, the guide RNA, or both are introduced into the population of microbial cells.

Introducing a Cas endonuclease/guide polynucleotide complex into the cell along with a donor DNA is typically necessary for generating a precise repair of the double strand break in the polynucleotide at the target site in the genome of the microbial cell. The components of the Cas system as provided herein can be introduced simultaneously or sequentially as desired by the user.

A. Introducing a Cas Endonuclease

For purposes of the present disclosure, introduction of a Cas endonuclease can be achieved in any convenient manner, including transfection, transduction, transformation, electroporation, particle bombardment, cell fusion techniques, and the like.

Alternatively, one can employ a Cas endonuclease that has nicking endonuclease activity (i.e., cleaves only one strand of DNA at the target site; also referred to herein as a “Cas nickase”) rather than double-strand break activity. Inducing nicks at the targets site does not activate the non-homologous end joining (NHEJ) pathway at the target site as would a double-stranded break, but it may improve homologous recombination between the genomic locus of interest (one that includes or is near to the target site for the Cas nickase) and the donor DNA. Examples of Cas nickases include Cas endonuclease variants as described below.

In certain embodiments, the Cas endonuclease (including, e.g., a Cas nickase) is a Cas9 endonuclease (see, e.g., PCT Publication No. WO2013/141680). Examples of Cas9 endonucleases include those from Streptococcus sp. (e.g., S. pyogenes, S. mutans, and S. thermophilus), Campylobacter sp. (e.g., C. jejuni), Neisseria sp. (e.g., N. meningitides), Francisella sp. (e.g., F. novicida), and Pasteurella sp. (e.g., P. multocida) (see, e.g., Cas9 endonucleases described in Fonfara et al., 2013). In some embodiments, the Cas endonuclease is encoded by an optimized Cas9 endonuclease gene, e.g., codon optimized for expression in a fungal cell.

In certain instances, the Cas endonuclease gene is operably linked to one or more polynucleotides encoding nuclear localization signals such that the Cas endonuclease/guide polynucleotide complex that is expressed in the cell is efficiently transported to the nucleus. Any convenient nuclear localization signal may be used, e.g., a polynucleotide encoding an SV40 nuclear localization signal present upstream (5′) of and in-frame (i.e., operably linked) with the Cas coding region and a polynucleotide encoding a nuclear localization signal derived from the T. reesei blr2 (blue light regulator 2) gene present downstream (3′) and in frame (i.e., operably linked) with the Cas coding region. Other nuclear localization signals can be employed.

In some embodiments, a Cas-expressing microbial cell is obtained by the user, and thus the user does not need to introduce a recombinant DNA construct capable of expressing a Cas endonuclease into the cell, but rather only need introduce a guide polynucleotide into the Cas expressing cell. For example, a fungal cell can first be stably transfected with a Cas expression DNA construct followed by introduction of a guide polynucleotide into the stable Cas expressing cell (either directly or using a guide polynucleotide expressing DNA construct). This set up provides certain advantages as the user can generate a population of stable Cas expressing fungal cells into which different guide polynucleotides can be introduced independently. In other embodiments, more than one guide polynucleotide can be introduced into the same Cas9 expressing cell.

As yet another example, a Cas endonuclease expressing host cell can be used to create a “helper strain” that can provide, in trans, the Cas endonuclease to a “target strain”. In brief, a heterokaryon can be created between the helper strain and the target strain, e.g., by fusion of protoplasts from each strain or by anastomosis of hyphae depending on the species of filamentous fungus. Maintenance of the heterokaryon will depend on appropriate nutritional and/or other marker genes or mutations in each parental strain and growth on suitable selective medium such that the parental strains are unable to grow, whereas the heterokaryon, due to complementation, is able to grow. Either at the time of heterokaryon formation or subsequently, a guide RNA and a donor DNA are introduced by transfection. The guide RNA may be directly introduced or introduced via a DNA construct having a Cas endonuclease expression cassette and a selectable marker gene. The Cas endonuclease is expressed from the gene in the helper strain nucleus and is present in the cytoplasm of the heterokaryon. The Cas endonuclease associates with the guide RNA to create an active complex that is targeted to the desired target site(s) in the genome, where the donor DNA is inserted. Subsequently, spores are recovered from the heterokaryon and subjected to selection or screening to recover the target strain with a donor DNA inserted at the target site. In cases in which an expression cassette is used to introduce the guide RNA, heterokaryons are chosen in which the guide RNA expression construct is not stably maintained.

In some embodiments, a Cas endonuclease is directly transfected in to the microbial cell. In other embodiments, a DNA vector comprising an expression cassette for the Cas endonuclease is transformed into a microbial cell.

i. A DNA Vector

A DNA construct comprising a nucleic acid encoding a Cas endonuclease can be constructed such that it is suitable to be expressed in a host cell. Because of the known degeneracy in the genetic code, different polynucleotides that encode an identical amino acid sequence can be designed and made with routine skills. It is also known that, depending on the desired host cells, codon optimization may be required prior to attempting expression.

A polynucleotide encoding a Cas endonuclease of the present disclosure can be incorporated into a vector. Vectors can be transferred to a host cell using known transformation techniques, such as those disclosed below.

A suitable vector may be one that can be transformed into and replicated within a host cell. For example, a vector comprising a nucleic acid encoding a Cas endonuclease of the present disclosure can be transformed and replicated in a bacterial host cell as a means of propagating and amplifying the vector. The vector may also be suitably transformed into an expression host, such that the encoding polynucleotide is expressed as a functional Cas endonuclease.

A representative useful vector is pTrex3gM (see, U.S. Patent Application Publication No. US 2013/0323798) and pTTT (see, U.S. Patent Application Publication No. 2011/0020899), which can be inserted into genome of host. The vectors pTrex3gM and pTTT can both be modified with routine skill such that they comprise and express a polynucleotide encoding a Cas endonuclease of the invention.

A vector useful for this purpose typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the Cas endonuclease to a host cell organelle such as the nucleus. For expression under the direction of control sequences, the nucleic acid sequence of the Cas endonuclease is operably linked to the control sequences in proper manner with respect to expression.

A polynucleotide encoding a Cas endonuclease of the present invention can be operably linked to a promoter, which allows transcription in the host cell. The promoter may be any DNA sequence that shows transcriptional activity in the host cell of choice and may be derived from genes encoding proteins either homologous or heterologous to the host cell, and genes that are inducible or constitutively expressed. Examples of promoters for directing the transcription of the DNA sequence encoding a Cas endonuclease, especially in a bacterial host, include the promoter of the lac operon of E. coli, the Streptomyces coelicolor agarase gene dagA or celA promoters, the promoters of the Bacillus licheniformis amylase gene (amyL), the promoters of the Bacillus stearothermophilus maltogenic amylase gene (amyM), the promoters of the Bacillus amyloliquefaciens amylase (amyQ), the promoters of the Bacillus subtilis xylA and xylB genes, and the like.

For transcription in a fungal host, examples of useful promoters include those derived from the gene encoding Aspergillus oryzae TAKA amylase, Rhizomucor miehei aspartic proteinase, Aspergillus niger neutral α-amylase, Aspergillus niger acid stable α-amylase, Aspergillus niger glucoamylase, Rhizomucor miehei lipase, Aspergillus oryzae alkaline protease, Aspergillus oryzae triose phosphate isomerase, Aspergillus nidulans acetamidase and the like. When a gene encoding a Cas endonuclease is expressed in a bacterial species such as an E. coli, a suitable promoter can be selected, for example, from a bacteriophage promoter including a T7 promoter and a phage lambda promoter. Along these lines, examples of suitable promoters for the expression in a yeast species include, but are not limited to, the Gal 1 and Gal 10 promoters of Saccharomyces cerevisiae and the Pichia pastoris AOX1 or AOX2 promoters. Expression in filamentous fungal host cells often involves cbh1, which is an endogenous, inducible promoter from T. reesei or constitutive glycolytic promoters (e.g., pki). For example, see Liu et al. 2008.

Typically Cas9 is not secreted for the purpose of the present invention. Rather Cas9 is targeted and retained in the nucleus such that the DNA editing occurs within the nucleus. In some embodiments, a Nuclear Localisation signal (NLS) may be added or fused to the Cas9 sequence. In other embodiments, however, for example, in bacteria, such a fusion or addition of NLS sequences is optional.

An expression vector may also comprise a suitable transcription terminator and, in eukaryotes, polyadenylation sequences operably linked to the DNA sequence encoding a Cas endonuclease. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., Published PCT Application No. WO 91/17243.

The procedures used to ligate the DNA construct encoding a Cas endonuclease, the promoter, terminator and other elements, respectively, and to insert them into suitable vectors containing the information necessary for replication, are known to persons skilled in the art and readily available. See, e.g., Sambrook et al., 2^{nd} ed., Cold Spring Harbor, 1989, and 3^{rd} ed., 2001.

ii. Method of Transformation

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) for transformation of Aspergillus strains. Genetically stable transformants can be constructed with vector systems whereby the nucleic acid encoding a Cas endonuclease is stably integrated into a host cell chromosome. Transformants are then selected and purified by known techniques.

The preparation of Trichoderma sp. for transformation, for example, may involve the preparation of protoplasts from fungal mycelia (e.g., see Campbell et al. 1989). The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

B. Introducing a Guide RNA

In some embodiments, introduction of the guide polynucleotide can be done in any convenient manner, including transfection, transduction, transformation, electroporation, particle bombardment, cell fusion techniques, etc.

In certain embodiments, a guide polynucleotide is introduced into the fungal cell by introducing a recombinant DNA construct that includes an expression cassette (or gene) encoding the guide polynucleotide. In some embodiments, the expression cassette is operably linked to a eukaryotic RNA pol III promoter. These promoters are of particular interest as transcription by RNA pol III does not lead to the addition of a 5′ cap structure or polyadenylation that occurs upon transcription by RNA polymerase II from an RNA pol II dependent promoter. In certain embodiments, the RNA pol III promoter is a filamentous fungal cell U6 polymerase III promoter.

As another example, a Cas endonuclease expressing host cell can be induced to uptake an in vitro synthesized guide RNA to enable Cas endonuclease activity and targeting to a defined site in the genome. In some cases, it will be desirable to induce uptake of both guide RNA and a separate DNA construct bearing a selectable marker gene to allow for selection of those cells that have taken up DNA and, at high frequency, are expected to have simultaneously taken up guide RNA. As above, screening those transformants that show an unstable phenotype with respect to the selectable marker for the genetic modification of interest (e.g., homologous recombination with a donor DNA) without vector DNA insertion is obtained.

For example, a Cas endonuclease expressing host cell can be transformed with a DNA construct including a guide RNA expression cassette containing a second selectable marker (and optionally a separate donor DNA). Host cells that are selected for using the second selectable marker will express the guide RNA from this DNA construct, which enables Cas endonuclease activity and targeting to a defined target site of interest in the genome.

In certain embodiments of the disclosure, the guide polynucleotide is a guide RNA that includes a crRNA region (or crRNA fragment) and/or a tracrRNA region (or tracrRNA fragment) of the type II CRISPR/Cas system that can form a complex with a type II Cas endonuclease. As indicated above, the guide RNA/Cas endonuclease complex can direct the Cas endonuclease to a microbial cell genomic target site, enabling the Cas endonuclease to introduce a double strand break into the genomic target site. In some cases, the RNA that guides the RNA/Cas9 endonuclease complex is a duplex that includes a crRNA and a separate tracrRNA. In other instances, the guide RNA is a single RNA molecule (e.g., a fusion) that includes both a crRNA region and a tracrRNA region (sometimes referred to herein as a fused guide RNA). One advantage of using a fused guide RNA versus a duplexed crRNA-tracrRNA is that only one expression cassette needs to be made to express the fused guide RNA

C. Introducing a Donor DNA

When a double-strand break is induced in the genomic DNA of a host cell (e.g., by the activity of a Cas endonuclease/guide RNA complex at a target site, the complex having double-strand endonuclease activity), the cell's DNA repair mechanism is activated to repair the break, which due to its error-prone nature, can produce mutations at double-strand break sites. The most common repair mechanism to bring the broken ends together is the non-homologous end-joining (NHEJ) pathway. The structural integrity of chromosomes is typically preserved by the repair, however deletions, insertions, or other rearrangements are possible (Siebert and Puchta, 2002; Pacher et al., 2007).

For target specific gene editing, e.g., gene insertion, gene replacement and other sequence integrations, a donor DNA includes a first region and a second region (i.e., homology arms) that are homologous to corresponding first and second regions in the genome of the fungal cell, wherein the regions of homology generally include or surround the target site at which the genomic DNA is cleaved by the Cas endonuclease. These regions of homology promote homologous recombination with their corresponding genomic regions of homology resulting in exchange of DNA between the donor DNA and the genome. As such, the provided methods result in the integration of the polynucleotide of interest of the donor DNA at or near the cleavage site in the target site in the fungal cell genome, thereby altering the original target site, thereby producing an altered genomic target site.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the fungal cell genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, such that the sequences undergo homologous recombination.

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

The lengths of the homology arms also contribute to transfection and recombination efficacy and efficiency. It is typically known in the art that such lengths range from 0.5 to 1 kb in order to achieve targeted editing.

It has been surprisingly found in the instant disclosure, that in certain filamentous fungi, homology arms as short as 100 bps or less (e.g., as short as 80 bps or less, as short as 60 bps or less, or even as short as 40 bps or less) in length can be used to achieve efficient homologous recombination stimulated by the guide RNA/Cas endonuclease complex. Moreover, while the traditional double stranded donor DNA (dsDNA) would work to mediate targeted gene editing, as exemplified herein single stranded donor DNA (ssDNA) of the instant disclosure performs equivalently in mediating homologous recombination stimulated by the guide RNA/Cas endonuclease complex, especially when shorter homologous arms are employed (i.e., homology arms as short as 100 bps or less). As such, the multi-step molecular manipulation and targeted gene editing mediated by the Cas system is substantially simplified.

D. Microbial Cells

Microbial cells employed in the methods and compositions disclosed herein may be any fungal host cells from the phyla Ascomycota, Basidiomycota, Chytridiomycota, and Zygomycota (as defined by Hawksworth et al., 1995) as well as the Oomycota (Hawksworth et al., 1995) and all mitosporic fungi (Hawksworth et al., 1995). In certain embodiments, the microbial host cells are yeast cells, e.g., Candida, Hansenula, Kluyveromyces, Pichia, Saccharomyces, Schizosaccharomyces, or Yarrowia cells. Species of yeast include, but are not limited to, Saccharomyces carisbergensis, Saccharomyces cerevisiae, Saccharomyces diastaticus, Saccharomyces douglasii, Saccharomyces kluyveri, Saccharomyces norbensis, Saccharomyces oviformis, Kluyveromyces lactis, and Yarrowia lipolytica.

In some embodiments, the microbial cells are filamentous fungal cells including, but not limited to, species of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma or Rasamsonia. In another preferred example, the filamentous fungal cells are selected from Aspergillus acufeatus, Aspergillus awamori, Aspergillus fumigatus, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, Trichoderma viride, Rasamsonia argillacea, Rasamsonia brevistipitata, Rasamsonia byssochiamydoides, Rasamsonia cylindrospora, Rasamsonia composticola, Rasamsonia eburnean or Rasamsonia emersonii.

In certain embodiments, the microbial host cells are bacterial cells, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis or a Streptomyces such as, e.g., a Streptomyces lividans or Streptomyces murinus or a gram negative bacterium, such as, e.g., an E. coli or a Pseudomonas sp.

For the aforementioned species, it is understood that the disclosure and source species would encompass both the perfect and imperfect states of such organisms, and other taxonomic equivalents thereof, e.g., anamorphs, regardless of the species name by which they are known. Those skilled in the art will readily recognize the identity of appropriate equivalents of such source species.

Strains of the above-mentioned species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

E. The Potential Target for Genome Editing

In some embodiments, specific genes are targeted for modification using the disclosed methods, including genes encoding enzymes (e.g., acetyl esterases, aminopeptidases, amylases, arabinases, arabinofuranosidases, carboxypeptidases, catalases, cellulases, chitinases, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, peroxidases, phenoloxidases, phytases, polygalacturonases, proteases, rhamno-galacturonases, ribonucleases, transferases, transport proteins, transglutaminases, xylanases, hexose oxidases, and combinations thereof).

There are numerous variations for implementing the methods described herein. For example, instead of having the Cas expression cassette present as an exogenous sequence in the fungal host cell, the Cas expression cassette can be integrated into the genome of the fungal host cell. Generating this parental cell line would allow a user to simply introduce a desired guide RNA (e.g., as a guide RNA expression vector) which would then target the genomic site of interest as detailed elsewhere herein. In some of these embodiments, the integrated Cas gene can be designed to include polynucleotide repeats flanking it for subsequent loop-out/removal from the genome if needed.

F. Implementing Gene Editing by a Cas Endonuclease/Guide Polynucleotide Complex

Virtually any site in a microbial cell genome may be targeted using the disclosed methods and compositions, so long as the target site includes the required protospacer adjacent motif, (hereinafter “PAM”). In the case of the S. pyogenes Cas9, the PAM has the sequence NGG (5′ to 3′; where N is A, G, C or T), and thus does not impose significant restrictions on the selection of a target site in the genome. Other known Cas9 endonucleases have different PAM sites (see, e.g., Cas9 endonuclease PAM sites described in Fonfara et al., 2013).

The length of at least one of the target sites can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic (i.e., the sequence on one strand reads the same in the opposite direction on the complementary strand). The cleavage site can be within the target sequence or the cleavage site can be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

In some cases, active variant target sequences in the genome of the fungal cell can also be used, meaning that the target site is not 100% identical to the relevant sequence in the guide polynucleotide (within the crRNA sequence of the guide polynucleotide). Such active variants can comprise at least 65%, 70%, 75%, 80%, 85%, 90%, 91%, 92%, 93%, 94%, 95%, 96%, 97%, 98%, 99% or more sequence identity to the given target site, wherein the active variant target sequences retain biological activity and hence are capable of being recognized and cleaved by a Cas endonuclease. Assays to measure the double-strand break of a target site by an endonuclease are known in the art and generally measure the overall activity and specificity of the agent on DNA substrates containing recognition sites.

Target sites of interest include those located within a region of a gene of interest. Non-limiting examples of regions within a gene of interest include an open reading frame, a promoter, a transcriptional regulatory element, a translational regulatory element, a transcriptional terminator sequence, an mRNA splice site, a protein coding sequence, an intron site, an intron enhancing motif, and the like.

G. Identifying a Microbial Cell with Desired Genome Editing

In certain embodiments, modification of the genome of the microbial cell results in a phenotypic effect that can be detected and, in many instances, is a desired outcome of the user. Non-limiting examples include acquisition of a selectable cell growth phenotype (e.g., resistance to or sensitivity to an antibiotic, gain or loss of an auxotrophic characteristic, increased or decreased rate of growth, etc.), expression of a detectable marker (e.g., fluorescent marker, cell-surface molecule, chromogenic enzyme, etc.), and the secretion of an enzyme the activity of which can be detected in culture supernatant.

When modification of the genome of the microbial cell results in a phenotypic effect, a donor DNA is often employed that includes a polynucleotide of interest that is (or encodes) a phenotypic marker. Any convenient phenotypic marker can be used, including any selectable or screenable marker that allows one to identify, or select for or against a fungal cell that contains it, often under particular culture conditions. Thus, in some aspects of the present invention, the identification of microbial cells having a desired genome modification includes culturing the microbial population of cells that have received the Cas endonuclease and guide polynucleotide (and optionally a donor DNA) under conditions to select for cells having the modification at the target site. Any type selection system may be employed, including assessing for the gain or loss of an enzymatic activity in the fungal cell (also referred to as a selectable marker), e.g., the acquisition of antibiotic resistance or gain/loss of an auxotrophic marker.

In some instances, the genomic modification in the microbial cells is detected directly using any convenient method, including sequencing, PCR, Southern blot, restriction enzyme analysis, and the like, including combinations of such methods.

The present disclosure is further described by the following examples which should not be construed as limiting the scope of the disclosure.

### EXAMPLES

Aspects of the present methods and compositions may be further understood in light of the following examples, which should not be construed as limiting. Modifications to materials and methods will be apparent to those skilled in the art.

### Example 1

### Preparation of Donor Templates

### Example 1.1

### Preparation of Single Strand Oligonucleotides

Twenty (20) nmole of single strand DNA fragments (ssDNAs) of various lengths: 70-nucleotides, 100-nucleotides and 200-nucleotides, including both upper and lower strands (SEQ ID NOs: 1-6), were produced by Integrated DNA Technologies (IDT) as lyophilized desalted DNA. These 70-nt, 100-nt and 200-nt single strand DNA fragments contained flanking sequences of about 23-24-nucleotides, 40-41-nucleotides, & 90-91-nucleotides, respectively. While the sequences listed below in this example are marked with the designation of “upper strain” or “lower strand”, it is believed such a designation is not strictly necessary in that a double-stranded break is made in this example, and either strand can function as a repair template. It is however believed that when Cas nickases are employed, such as described herein, the efficiency of repair would depend on the strand used as repair template.

-Single upper strand oligonucleotides of 70 bases
SEQ ID NO: 1
GTCGTGCTCAAGACGCACTACGACGTTTAAACCTTAATTAAGCATGGTCT
CGGGCTGGGACTTCCACCCG
-Single lower strand oligonucleotides of 70 bases
SEQ ID NO: 2
CGGGTGGAAGTCCCAGCCCGAGACCATGCTTAATTAAGGTTTAAACGTCG
TAGTGCGTCTTGAGCACGAC
-Single upper strand oligonucleotides of 100 bases
SEQ ID NO: 3
AAGATTGGCCCGTCGATTGTCGTGCTCAAGACGCACTACGACGTTTAAAC
CTTAATTAAGCATGGTCTCGGGCTGGGACTTCCACCCGGAGACGGGCACG
-Single lower strand oligonucleotides of 100 bases
SEQ ID NO: 4
CGTGCCCGTCTCCGGGTGGAAGTCCCAGCCCGAGACCATGCTTAATTAAG
GTTTAAACGTCGTAGTGCGTCTTGAGCACGACAATCGACGGGCCAATCTT
-Single upper strand oligonucleotides 200 bases
SEQ ID NO: 5
GCCTGAGCGCCGACGTGCCGACAGCGCGCGAGCTGCTGTACCTGGCCGAC
AAGATTGGCCCGTCGATTGTCGTGCTCAAGACGCACTACGACGTTTAAAC
CTTAATTAAGCATGGTCTCGGGCTGGGACTTCCACCCGGAGACGGGCACG
GGAGCCCAGCTGGCGTCGCTGGCGCGCAAGCACGGCTTCCTCATCTTCGA
-Single lower strand oligonucleotides 200 bases
SEQ ID NO: 6
TCGAAGATGAGGAAGCCGTGCTTGCGCGCCAGCGACGCCAGCTGGGCTCC
CGTGCCCGTCTCCGGGTGGAAGTCCCAGCCCGAGACCATGCTTAATTAAG
GTTTAAACGTCGTAGTGCGTCTTGAGCACGACAATCGACGGGCCAATCTT
GTCGGCCAGGTACAGCAGCTCGCGCGCTGTCGGCACGTCGGCGCTCAGGC

### Example 1.2

### Preparation of Double Strand Oligonucleotides

Double stranded donor DNA templates of various lengths: 330 bps, 730 bps, and 1100 bps, were produced by Integrated DNA Technology (IDT) as 200 ng DNA fragments.

Each of the double strand donor templates contained a 19-nucleotide insertion sequence that was used to replace the entire target site TS2 sequence (SEQ ID NO: 10).

The double strand donor template of 330 bps (SEQ ID NO: 7) has 152 bps of upstream flanking sequence and 159 bps of downstream flanking sequence.

The double strand donor template of 730 bps (SEQ ID NO: 8) has 352 bps of upstream flanking sequence and 359 bps of downstream flanking sequence.

The double strand donor template of 1100 bps (SEQ ID NO: 9) has 562 bps of upstream flanking sequence and 529 bps of downstream flanking sequence.

Insertion sequence between the upstream and downstream flanking sequences contained 3 stop codons in 3 reading frames and the restriction cleavage sites Pme1 & Pac1.

### Example 2

### Target Site Selection and sgRNA Synthesis

### Example 2.1

### Target Site Selection

The pyr 4 marker gene was selected to test homologous repair using CRISPR-Cas9 system in the presence of exogenous donor templates including single strand oligonucleotides and double strand DNAs, as described in Example 1 above. The target site with the motif G-N20-GG was selected with the 23-bp sequence of SEQ ID NO: 10, which included the PAM (TGG) site.

The template for sgRNA synthesis was produced by IDT as a DNA fragment with the sequence set forth as SEQ ID NO: 11, which contains the T7 promoter sequence followed by the 20-base target site (in italic and underlined text) without the PAM site, guide RNA scaffold, followed by the terminator site (TTTTT).

-Template for guide RNA sequence
SEQ ID NO: 11
CGCGAAATTAATACGACTCACTATAGGGCTCAAGACGCACTACGACAGTT
TTAGAGCTAGAAATAGCAAGTTAAAATAAGGCTAGTCCGTTATCAACTTG
AAAAAGTGGCACCGAGTCGGTGCTTTTTAGC

### Example 2.2

### Production of sqRNA

Guide RNAs were produced in vitro using the MEGA shortscript™ kit (Ambion, Product No. AM 1354). In vitro transcription was carried out at 37° C. for at least 5 hours. The resulting RNA was purified using Qiagen RNAeasy Plus mini kit (Qiagen). Nano drop was used to determine the amount of RNA produced.

### Example 3

### Transformation of Guide RNAs and Single Strand Template

A Trichoderma reesei strain was derived from RL-P37 by screening for increased cellulase productivity and having a single point mutation that inactivates the pyr2 gene making the strain a uridine auxotroph. This strain was transformed with a DNA construct containing an expression cassette for Streptococcus pyogenes Cas9 under the control of the pyruvate kinase (pki) promoter and an expression cassette for the pyr2 gene from T. reesei under the control of the its native promoter as described in PCT International Application No. PCT/CN2014/093918. A transformant with the Cas9-pyr2 cassette integrated into the genome and constitutively expressing the Cas9 gene was identified by selecting for cells having a functional pyr2 gene (growth without uridine supplementation on Vogels media).

Protoplasts were prepared from the T. reesei strain. The strain was grown on PDA plate for 5 days at 30° C. Spores were collected and inoculated into 50 mL of YEG (5 g/L yeast extract plus 20 g/L glucose) broth in a 250 mL, 4-baffle shake flask, and incubated at 37° C., for 16-20 hours at 200 rpm.

The mycelia were recovered by transferring the liquid volume into 50 mL conical tubes and spinning at 2,500 rpm for 10 minutes. The supernatant was decanted. The mycelial pellet was then transferred into a 250 mL, 0.22 micron CA Corning filter bottle with 40 mL solution containing 2g lysing enzyme (SIGMA). The mixture was thereafter incubated at 30° C., mixing at 200 rpm, for 2 hours to generate protoplasts for transformation.

Protoplasts were harvested by filtration through sterile miracloth into a 50 mL conical tube. They were then pelleted by spinning at 2,000 rpm for 5 minutes and aspirated. The protoplast pellet was washed once with 50 mL of 1.2 M sorbitol, spun down, aspirated, and washed again with 25 mL of sorbitol/CaCl₂.

Protoplasts were counted and then pelleted at 2,000 rpm for 5 minutes, the supernatant was decanted, and the protoplast pellet re-suspended in an amount of sorbitol/CaCl₂ sufficient to generate a protoplast concentration of 1.25×10⁸ protoplasts per mL, generating a protoplast solution.

Aliquots of up to 15 μg of in vitro synthesized guide RNA (at 1.2μg/μL) and 2 μL of 0.1 mM single strand DNA template were placed into 15 mL conical tubes and the tubes were put on ice. Then 200 μL of the protoplast suspension was added along with 50 μL PEG solution to each transformation aliquot. The tubes were mixed gently and incubated on ice for 20 minutes. A PEG solution, in the volume of 4 mL, was added to the transformation aliquot tubes, and these were incubated at room temperature for 5 minutes. Sorbitol/CaCl₂ solution, in the volume of 3.5 mL, was added to the tubes (generating a total volume of 8 ml). The transformation mixture was divided into 2 aliquots each containing about 4 mL. Vogels sorbitol containing 1.0 mg/mL uridine melted top agar (kept molten by holding at 50° C.) was mixed with the transformation reaction, which was then plated, and incubated at 30° C. for 4-5 days.

Selection was carried out using an overlay agar consisting of Vogels with 1.8 mg/mL FOA (Zymo Research Corp.) and 0.5 mg/mL uridine. Controls were carried out using protoplasts incubated with 2 μL of 0.1 mM single strand DNA template without the guide RNA.

### Example 4

### Characterization of FOA Resistant Transformants

Genomic DNA was isolated from T. reesei colonies growing on FOA-uridine plates using the hot phenol extraction protocol. Small amount of mycelia (˜0.25 cm² mycelia with agar) were transferred to 600 μL eppendorf tubes, grounded and re-suspended in 120 μL Lysis buffer. A lysis buffer was prepared by blending 200 μL of 1M Tris, at pH8, 200 μL of 3M sodium acetate pH5.8, and 200 μL of phenol:chloroform:isoamyl alcohol blend (25:24:1, v/v), and 1,800 μL of a TE buffer prepared with 10 mM Tris-HCl, pH 8, and 0.1 mM EDTA. Chloroform (120 μL) was subsequently added to the lysed mycelia, mixed by vortexing and incubated in a thermomixer for 6 minutes at 72° C. The lysate was then mixed briefly and centrifuged for 3 minutes at maximum speed. The aqueous phase was transferred to a tube containing 100 μL of isopropanol, mixed and centrifuged for 10 minutes. The pellet was washed with 1 mL 70% ethanol and centrifuged. The pellet was re-suspended in 60 μL TE buffer, incubated at 72° C. and used as template for PCR.

PCR reactions were carried out in 25 μL reaction volume using 1 μL of genomic DNA, 0.1 μL each of 50 μM primers (forward & reverse primer), 0.25 μL of PCR nucleotide mix and 0.25 μL PfuUltra II DNA polymerase (Agilent Technologies).

The following primers were used to PCR across the pyr4 target site TS2. Primer pairs with:

SEQ ID NO: 12-1F:
5′-CCATCTTGGCTGACGAAAAAGGTCTG-3′;
and
SEQ ID NO: 13-1R:
5′-CATGCAAAGATACACATCAATCGCAGCTG-3′.

These primers were used when using the single strand DNA as donor template.

Primer pairs:
-MH179
SEQ ID NO: 14
5′-CATCGACTACTGCTGCTCTGCTC-3′;
and
-MH180
SEQ ID NO: 15
5′ATCGCAGCTGGGGTACAATCATC-3′

were used to PCR colonies from transformations using the double strand DNA as donor templates. PCR products were analyzed by electrophoresis using 0.8% agarose gels. PCR products were purified using Qiaquick PCR Purification kit (Qiagen; Catalogue No. 28104).

DNA sequencing was carried out by Sequetech Corp (Moutainview, Calif.) using sequencing primers μMH094 (SEQ ID NO:16)

Restriction digestions were carried out for 15 minutes at 37° C. in 20 μL volume using 5 μL of PCR product and restriction enzymes from New England Biolabs (Pac1, Catalog No. R5547S, Pme1, Catalogue No. R0560S). Reaction products were analyzed by electrophoresis on a 1% agarose gel.

### Example 5

### CAS9-Mediated Homology Directed Repair Using Single Strand Oligonucleotides of 100 Nucleotides as Donor DNA Template

The donor DNA template (100-nucleotides) included two single strand DNAs: (1) SEQ ID NO:4, which is the lower strand (and is the strand complementary to target site TS2 of SEQ ID NO:10); and (2) SEQ ID NO: 3, which is the upper strand and is the same strand as TS2.

SEQ ID NO: 3 and 4 each included a 19-nucleotide stop codon sequence flanked by a 40-nucleotide (5′) homology arm and 41-nucleotide (3′) homology arm (as shown in FIG. 1).

A single strand DNA of 70 bases (SEQ ID NOs: 1 & 2) was also designed with homology arms of 23-24 nucleotides flanking the 19-nucleotide insertion sequence, and 200 bases (SEQ ID NOs: 5 & 6) with homology arms of 90-91 nucleotides flanking the 19-nucleotide insertion sequence.

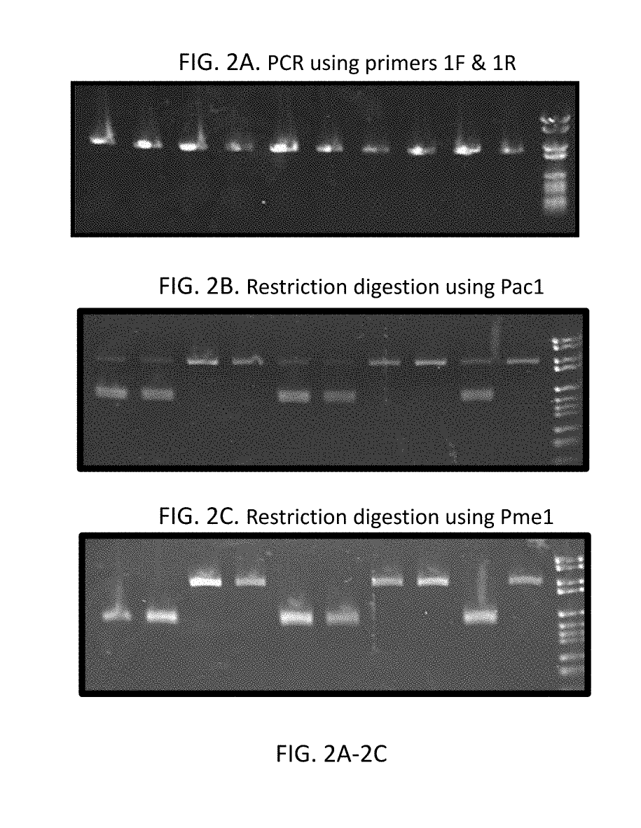

In 5 out of 10 FOA resistant strains that were isolated, a 1,200-bp PCR product containing the restriction sites Pme1 & Pac1 was obtained and sequenced. The efficient disruption and repair was observed in 50% of the FOA resistant colonies using single strand oligonucleotides of 100 bases with a 40-nucleotide 5′ homology arm and 41-nucleotide 3′ homology arm flanking the stop codon insertion at the pyr4 gene locus (shown in FIG. 2).

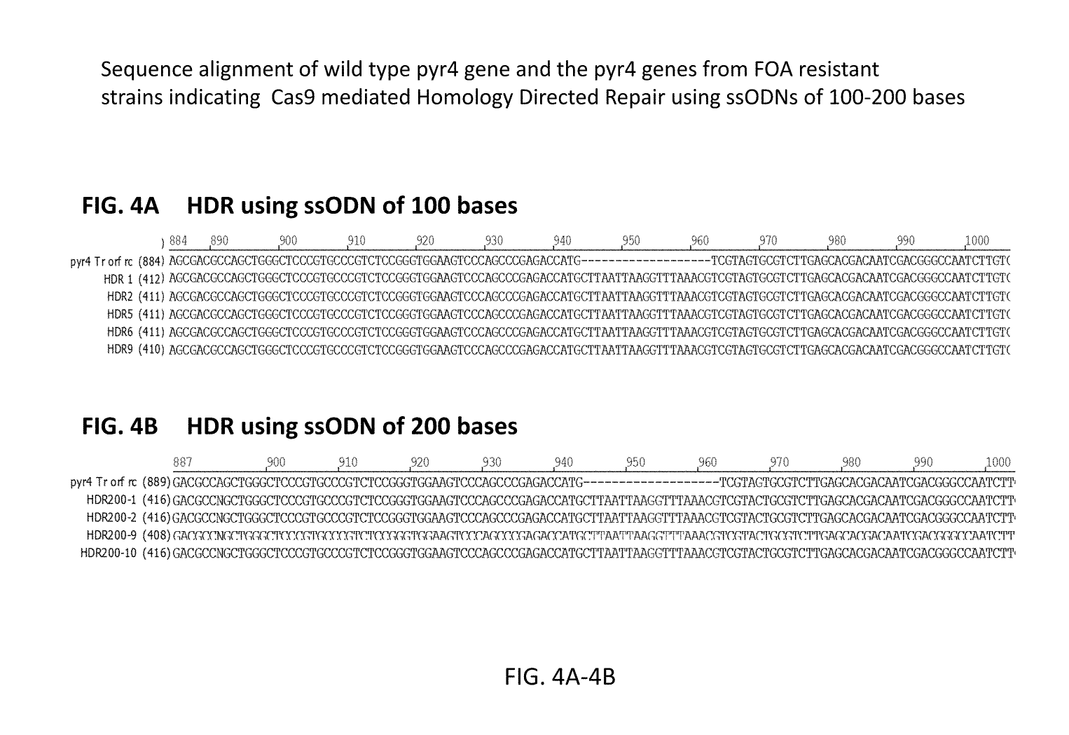

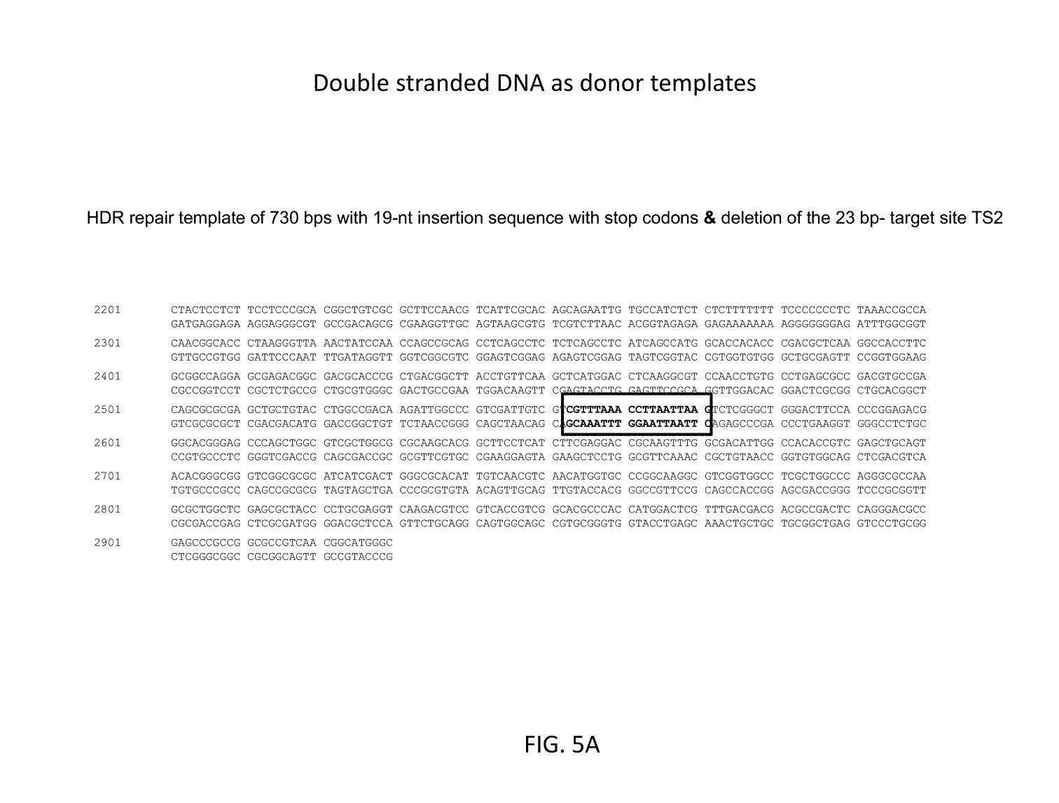

DNA sequence alignments are depicted in FIG. 4A, using single strand DNA as donor template for repairing by homologous recombination (HR). The PCR products amplified from FOA resistant strains were purified and sequenced using SEQ ID NO:16. Sequence analysis of individual repair events revealed that the pyr4 gene contains the single strand DNA repair template in 5 out of 10 clones.

The remaining 5 clones contained indels, indicating site-specific induction of DSBs and repairing via the non-homologous end joining pathway (NHEJ). The sequencing result was consistent with restriction digestion results in FIG. 2.

PCR products digested by Pac1 & Pme1 indicated that homologous recombination via the homology directed repair (HDR) pathway has occurred and led to the presence of the 19-nucleotide insertion sequence with 2 restriction sites.

### Example 6

### CAS9-Mediated Homology Directed Repair Using Single Strand Oligonucleotides of 200 Bases as Donor DNA Template

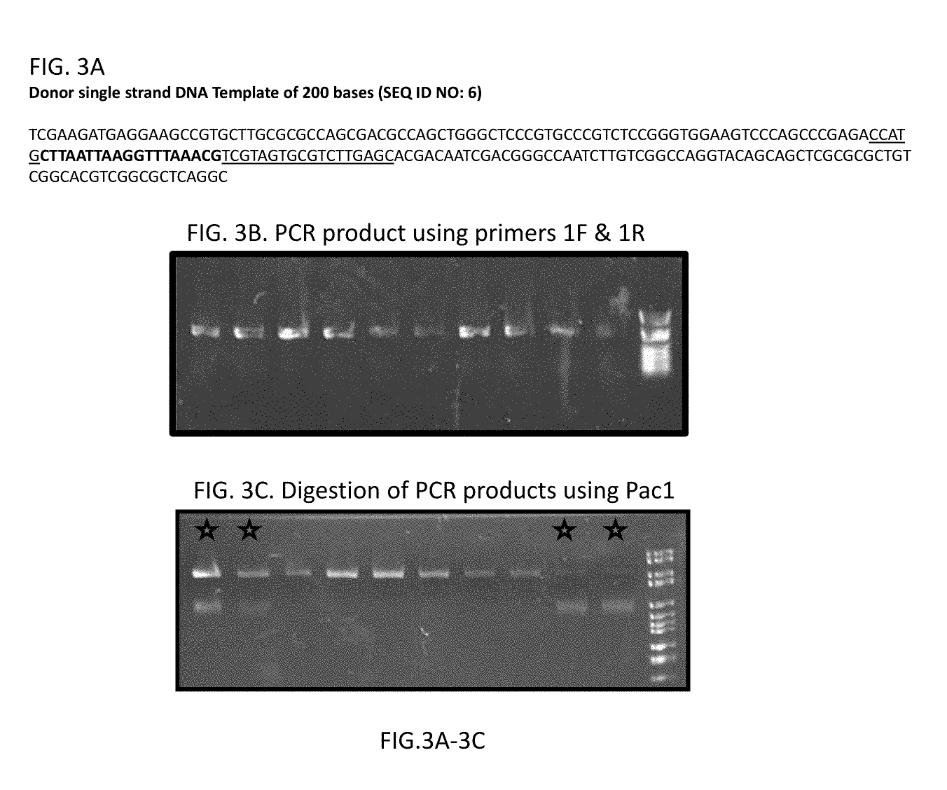

Mycelia from 10 FOA resistant colonies were isolated and genomic DNA extractions were carried out. The results are shown in FIG. 3.

The sequence of the 200 bases single strand DNA (SEQ ID NO: 6) was used in transformation experiments. Agarose gel electrophoresis (0.8% agarose gel) was used to analyze the PCR product (1.2 kb band) derived using 1F & 1R primers (SEQ ID NO: 12 & 13). Restriction digestion using Pac1 revealed the presence of the Pac1 site in 4 out of 10 PCR products. A frequency of homology directed repair was observed in 40% of the strains (as shown in FIG. 3).

All 10 PCR products were purified and subjected to DNA sequencing using sequencing primer SEQ ID NO: 16. FIG. 4B presents the results of such sequencing and analyses of individual repair events, which revealed that the pyr4 gene contained the single strand DNA repair template in 4 out of 10 clones.

The remaining 6 clones contained indels, indicating Cas9 induced DSBs and executed repair using the non-homologous end joining pathway (NHEJ).

The sequencing result was consistent with restriction digestion results as shown in FIG. 3. These results confirmed that the stimulation of homology-directed repairs (HDR) in the presence of the 200-nucleotide single strand DNA donor template in addition to the NHEJ repair pathways, induced by the Cas9 mediated double strand breaks. In conclusion, homology flanking sequences of 90-91 bases are able to stimulate repair by HDR in fungi.

### Example 7

### CAS9 Mediated Homology Directed Repair Using Double Strand DNA as Donor DNA Template

Double strand DNA templates have traditionally been used in gene replacement experiments with 500 bps as the minimum length of flanking homology sequences. The goal of this example is to test whether the double strand DNA template with the shorter flanking homology sequences from 150 bps to up to 500 bps (SEQ ID NOs: 7, 8 and 9) can also induce homologous recombination.

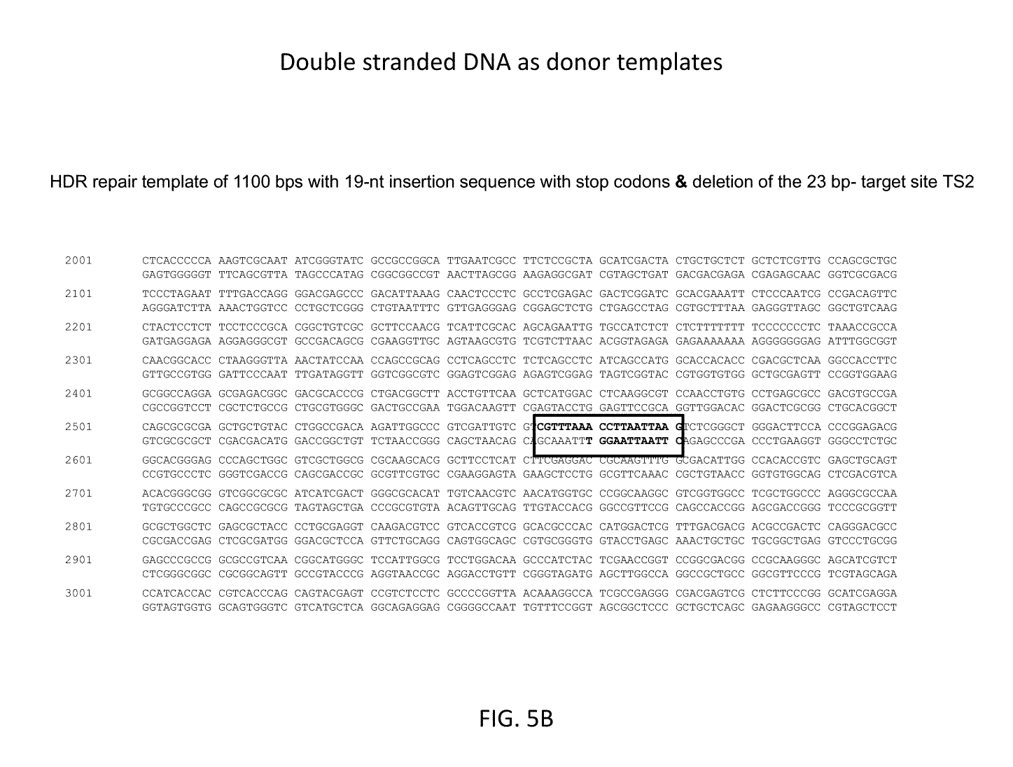

FIG. 5 depicts the double strand DNA templates used, each comprising insertion codons and the almost symmetrical flanking homologous arms.

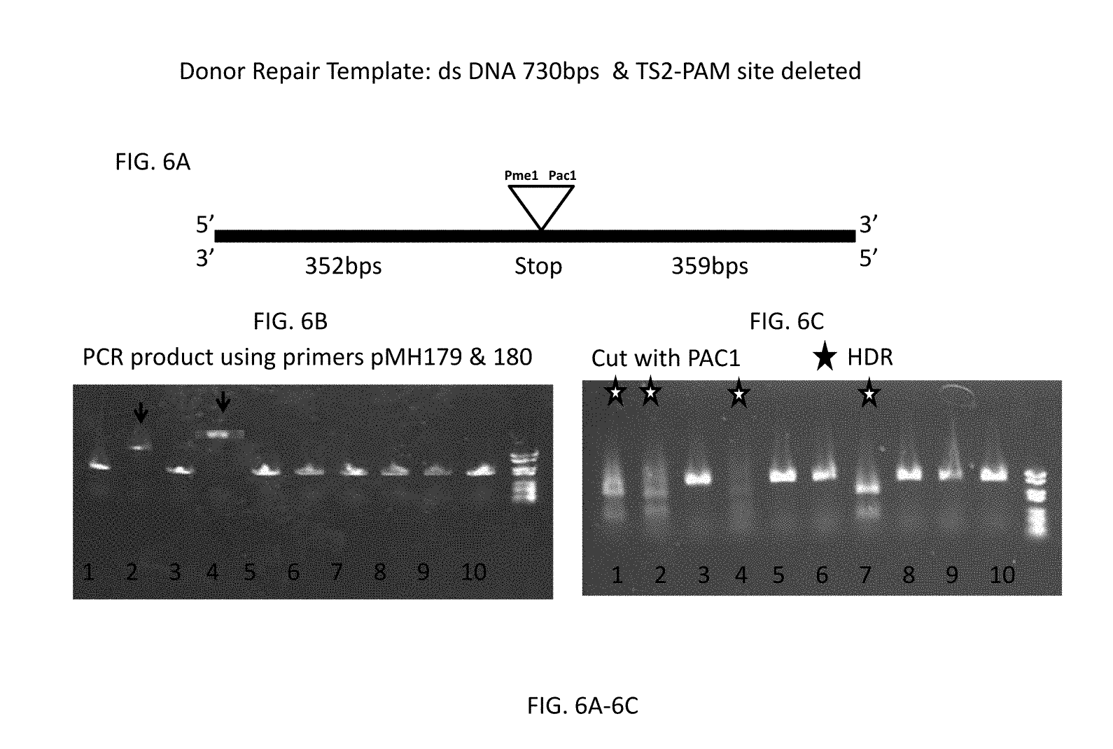

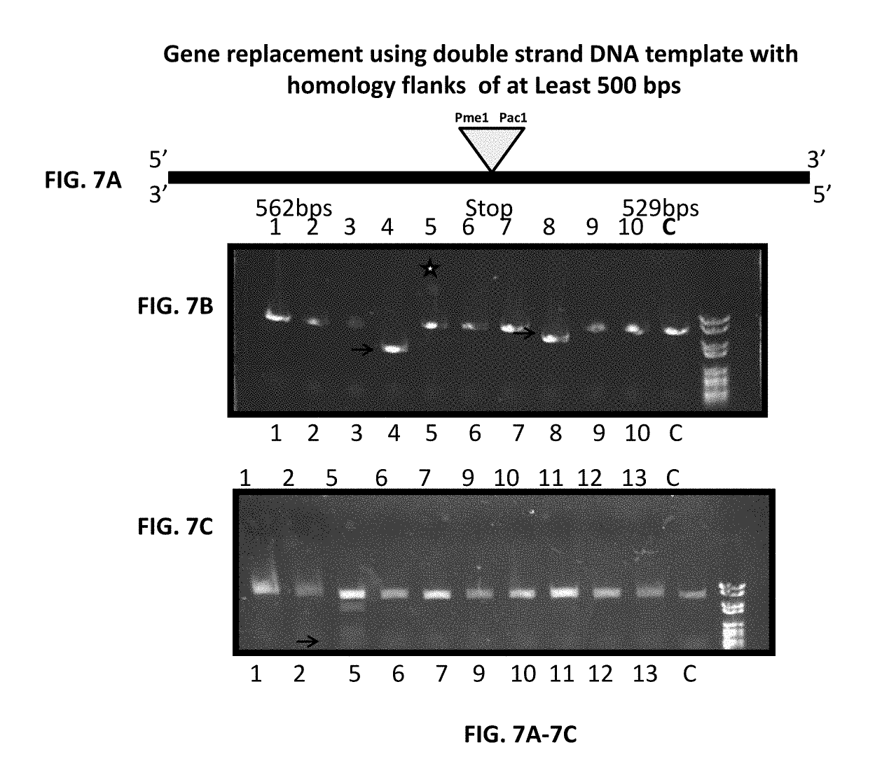

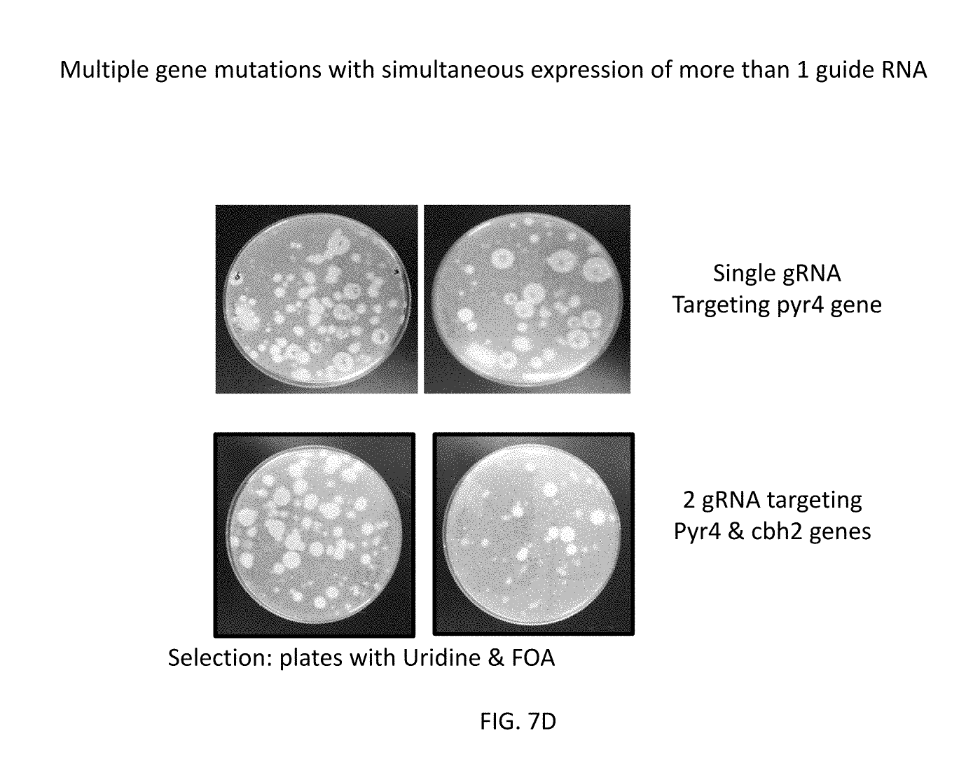

Agarose gel electrophoresis of PCR products indicated the presence of an unusually higher molecular weight bands faintly visible in Gel B of FIG. 6, lanes 2 and 4. This might have reflected aberrant repair events, possibly caused by concatamerization of the donor template or non-specific recombination products.

In Gel C, restriction enzyme digestion of the PCR products indicated the presence of the Pac1 site within the pyr4 gene. This was observed in 4 out of 10 products, indicating that 40% of the experiments/colonies had Cas9 mediated homology directed repair and 60% of the experiments/colonies had NHEJ-related repair.

Gene replacement experiments were conducted using a 1,100-bp double strand donor template with symmetrical homology arms of about 500 bps. The donor DNA also contained a stop insertion with restriction sites, which was used for diagnosing and confirming FOA resistant colonies.

Agarose gel analysis was performed on each of the PCR products amplified from genomic DNA of individual FOA resistant colonies.

As indicated in FIG. 6, agarose gel B, lanes 4 and 8 indicated PCR products with low molecular weights as compared to those products from the control sample (C), indicating large deletions in the pyr4 gene. Restriction digestions with Pac1 demonstrated that, aside from sample #5 (as shown in Agarose gel C lane 5), a majority of the PCR products were not digested or digestable with Pac1. This indicated that HDR occurred at a low frequency whereas the NHEJ repair pathway occurred predominantly.

### Example 8

### Homology Directed Repair Using CRISPR-CAS9 & Donor DNA Templates (ssODN) in Aspergillus

An Aspergillus tubigensis overproducing strain 3M-43/pyrA (see, Nikolaev et al., 2013) was used to conduct genomic editing, with the goal of disrupting the L-arabitol dehydrogenase (LDA) pathway, using Cas9. As described in Nikolaev et al. (2013), disruption of the L-arabitol dehydrogenase gene pathway was expected to lead to an increased xylanase production by that strain when cultivated under suitable conditions.

Nikolaev et al. (2013) further reports that increased xylanase production from the LDA-disrupted Aspergillus tubigensis strain 3M-43 is mediated by the transcription factor xlnR; and that a single resulting strain out of 80 attempted contained a disrupted xlnR gene using the conventional gene replacement strategy, indicating that the conventional gene replacement strategy (while workable) was inefficient and lacked robustness at best.

In the present example, ultramers of 100-200 bases in both upper and lower strands can be purchased from Integrated DNA Technologies (IDT). Four (4) different single strand oligonucleotides having SEQ ID NOs: 17-20 can also be made by IDT, to be used as donor templates for homology directed repair of the Cas9 induced double strand breaks.

More particularly, SEQ ID NO:17 is a 100-base ultramer upper strand with the 19-base stop codon insertion (in uppercase), with homology arms of 44 bases at the 5′ end and 37 bases at the 3′end:

5′-
atcagccgtgcgtgtgaccagtgtaaccaactccgaacgaaatgCGTTTA
AACCTTAATTAAGcgacgggcagcat
ccgtgcgctcattgcattggtagg-3′

SEQ ID NO: 18 is a 100-base ultramer lower strand with the 19-base stop codon insertion (in uppercase) with homology arms of 37 bases at the 5′ end and 44 bases at the 3′end:

5′-
cctaccaatgcaatgagcgcacggatgctgcccgtcgCTTAATTAAGGTT
TAAACGcatttcgttcggagttggttacactggtcacacgcacggctga
t-3′

SEQ ID NO:19 is a 200-ultramer upper strand with the 19-base stop codon insertion (in uppercase) with homology arms of 94 bases at the 5′ end and 87 bases at the 3′ end:

5-′ctcgctctgccgtccgcaaaacctcgtcttcagctccggttcgccgc
cgaatcagccgtgcgtgtgaccagtgtaaccaactccgaacgaaatgCGT
TTAAACCTTAATTAAGcgacgggcagcatccgtgcgctcattgcattggt
aggcttccgctctttctccgatgccggcgatgaggcggacgcttgactga
cct-3′

SEQ ID NO:20 is a 200-ultramer lower strand with the 19-base stop codon insertion (in uppercase) with homology arms of 87 bases at the 5′ end and 94 bases at the 3′ end:

5′-Aggtcagtcaagcgtccgcctcatcgccggcatcggagaaagagcgg
aagcctaccaatgcaatgagcgcacggatgctgcccgtcgCTTAATTAAG
GTTTAAACGcatttcgttcggagttggttacactggtcacacgcacggct
gattcggcggcgaaccggagctgaagacgaggttttgcggacggcagagc
gag-3′

As presented above, the oligonucleotides that are 100 bases long can contain a 19 bases long stop codon flanked by 5′ and 3′ homology arms of 44 and 37 bases (SEQ ID NOs: 17 and 18), respectively.

The oligonucleotides that are 200 bases long can also contain the 19-base stop codon insertion flanked by homology arms of 94 and 87 bases at the 5′ and 3′, respectively (SEQ ID NOs: 19 and 20).

A Cas9 expression vector pGdpA:Cas9 can be constructed using the codon optimized Cas9 gene (i.e., codon optimized for Trichoderma reesei expression), as provided herein). The Aspergillus nidulans glyceraldehyde-3-phosphate dehydrogenase gene (gpdA) promoter, the 5′ untranslated region of gpdA mRNA, and the Aspergillus nidulans trp C terminator can be used to drive the expression of the Cas9 encoding sequence. The 3.9 kg Xba1 fragment of the Aspergillus niger pyrA gene can be inserted into the pGpd:Cas9 plasmid, to be used as a selection marker.

Fungal co-transformation can be carried out using 2 μg of the Cas9 expression vector thus constructed, with pyrA selection, 20 μg of in vitro synthesized guide RNA and 100 μM of either a 100- or a 200-base single strand ultramer, containing the stop codon of SEQ ID NO:21 in three reading frames: SEQ ID NO: 21(CGTTTAAACCTTAATTAAG)

The xlnR gene encodes a zinc binuclear cluster Zn2Cys6 protein. A 20-bp target sequence (SEQ ID NO:22) can be chosen: SEQ ID NO:22: (CAACTCCGAACGAAATGCGA). SEQ ID NO:22 precedes the PAM site “CGG” as the target site for Cas9 induced double strand break, as it is located within the zinc binuclear DNA binding domain near the N-terminus of xlnR.

A template sequence for in vitro synthesis of the guide RNA containing the T7 promoter (underlined), the 20-bp target site (uppercase), the tracr sequence (SEQ ID NO:23) can be ordered as gblocks from IDT and the guide RNAs can then be synthesized in vitro using the Megashort Script Kit (Ambion).

SEQ ID NO: 23 is a template for gRNA synthesis in vitro:

cgcgaaattaatacgactcactataggCAACTCCGAACGAAATGCGAgtt
ttagagctagaaatagcaagttaaaataaggctagtccgttatcaacttg
aaaaagtggcaccgagtcggtgctttttttaa

Co-transformation can be carried out by preparing a transformation mixture with protoplasts from Aspergillus tugingensis 3M-43/pyrA strain and the Cas9 expression vector with pryA selection as described above, at an amount of 2 μg, the in vitro synthesized guide RNA, at the amount of 20 μg, and a 100-base or 200-base single strand ultramer donor template. The transformation mixture thus prepared is then plated onto minimal media plates containing per liter, 6 g NaNO₃, 1.5 g KH₂PO₄, 0.5 g MgSO₄.7H₂O, 0.5 g KCl, Vishniac trace elements, 1.5% agar, and 20 g fructose as a carbon source (pH 6.0).

The colonies that appear on the minimal agar plates following inducation are then transferred to a new minimal agar plate containing D-xylose and xylan as carbon sources.

Several transformants will demonstrate reduced growth on D-xylose or even complete absence of xylanase activity, as can be assayed or estimated based on the halo formation on xylan-based agar plates. The reduced growth or complete absence of xylanase activity are good indicators that the disruption of transcription factor is successful.

Colonies can also be screened for endoxylanase (EXL) activity after growth in liquid culture on 3% sugar beet pulp (SBP) substrate and wheat bran (WB) for 5 days at 34° C. Mutation(s) in the xlnR gene can be confirmed by the absence of xylanase activity. Such a deletion or mutation can also be confirmed with PCR using genomic DNA and xlnR gene specific primers of SEQ ID NO: 24 (forward primer) and SEQ ID NO: 25 (reverse primer), in colonies manifesting the xlnR gene knockout phenotype. PCR products of about 3,820 kb in size can be generated in all transformants. Those transformants with reduced growth on D-xylose and absence of xylanase activity will show PCR products containing the pme1 and pac1 t sites. Restriction digestion can be used to confirm the presence of the sites, resulting in 2 bands of about 1,100 bp and 2,700 bp, in size. These bands would confirm the successful Cas9-induced double strand breaks and homology directed repair using single strand oligonucleotides acting as donor repair templates in Aspergillus tubigensis.

The results will also demonstrate that using the CRISPR-Cas9 strategy is a much more efficient means to achieve gene editing in a filamentous fungal strain such as an Aspergillus, as compared to the conventional means of modifying or disrupting genes in fungal strains.

### Example 9

### Expression of CAS9 in Bacillus Subtilis

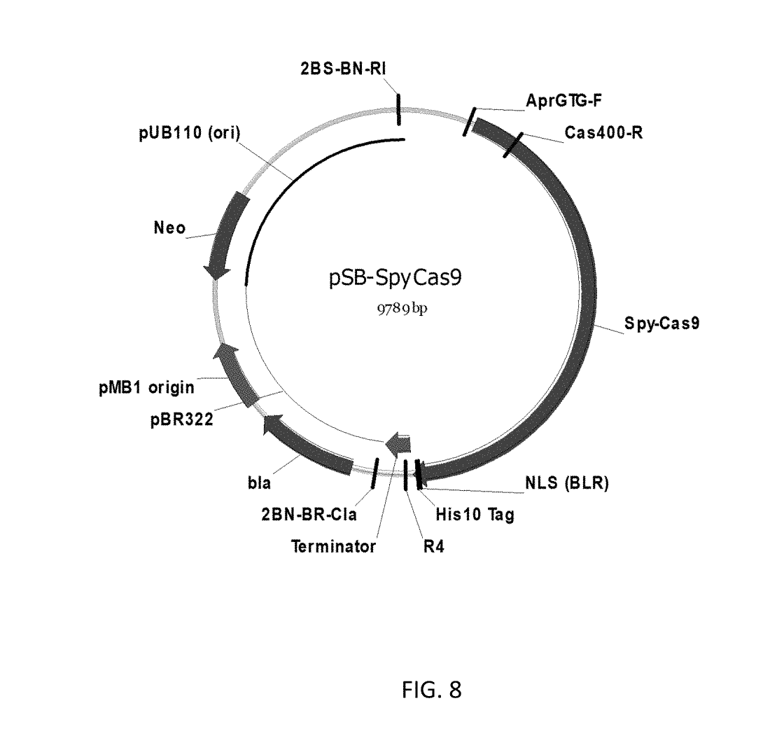

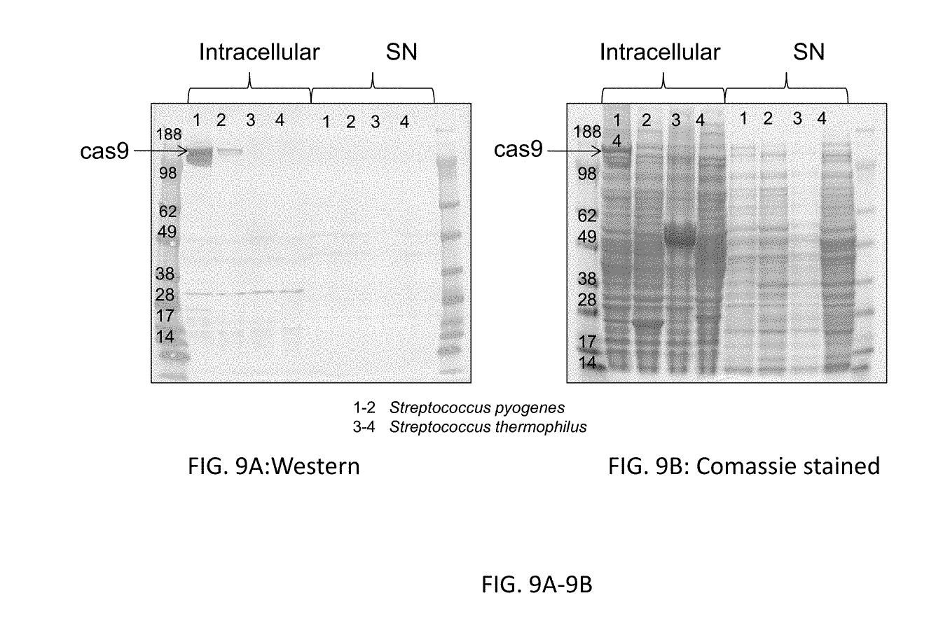

1. Construction of pSB-SpyCas9 Expression Vector Set Forth in FIG. 8

The SpeI-HindIII fragment (4.2 kb) carrying the SpyCas9 gene (SEQ ID NO: 26) was ligated into pSB cut with the same enzymes (resulting a fragment of 5.6 kb). More particularly, the polynucleotide of SEQ ID NO:26 is a sequence of Cas9 of Streptococcus pyogenes M1 GAS (Locus Spy_1046), with the NdeI-XhoI fragment, and the BsrDI restriction site marked by bold texts. The C-terminal underlined texts mark the nuclear localization sequence and deca-His tag.

CATATGGCCCCAAAAAAGAAACGCAAGGTTATGGATAAAAAATACAGCAT
TGGTCTGGATATCGGAACCAACAGCGTTGGGTGGGCAGTAATAACAGATG
AATACAAAGTGCCGTCAAAAAAATTTAAGGTTCTGGGGAATACAGATCGC
CACAGCATAAAAAAGAATCTGATTGGGGCATTGCTGTTTGATTCGGGTGA
GACAGCTGAGGCCACGCGTCTGAAACGTACAGCAAGAAGACGTTACACAC
GTCGTAAAAATCGTATTTGCTACTTACAGGAAATTTTTTCTAACGAAATG
GCCAAGGTAGATGATAGTTTCTTCCATCGTCTCGAAGAATCTTTTCTGGT
TGAGGAAGATAAAAAACACGAACGTCACCCTATCTTTGGCAATATCGTGG
ATGAAGTGGCCTATCATGAAAAATACCCTACGATTTATCATCTTCGCAAG
AAGTTGGTTGATAGTACGGACAAAGCGGATCTGCGTTTAATCTATCTTGC
GTTAGCGCACATGATCAAATTTCGTGGTCATTTCTTAATTGAAGGTGATC
TGAATCCTGATAACTCTGATGTGGACAAATTGTTTATACAATTAGTGCAA
ACCTATAATCAGCTGTTCGAGGAAAACCCCATTAATGCCTCTGGAGTTGA
TGCCAAAGCGATTTTAAGCGCGAGACTTTCTAAGTCCCGGCGTCTGGAGA
ATCTGATCGCCCAGTTACCAGGGGAAAAGAAAAATGGTCTGTTTGGTAAT
CTGATTGCCCTCAGTCTGGGGCTTACCCCGAACTTCAAATCCAATTTTGA
CCTGGCTGAGGACGCAAAGCTGCAGCTGAGCAAAGATACTTATGATGATG
ACCTCGACAATCTGCTCGCCCAGATTGGTGACCAATATGCGGATCTGTTT
CTGGCAGCGAAGAATCTTTCGGATGCTATCTTGCTGTCGGATATTCTGCG
TGTTAATACCGAAATCACCAAAGCGCCTCTGTCTGCAAGTATGATCAAGA
GATACGACGAGCACCACCAGGACCTGACTCTTCTTAAGGCACTGGTACGC
CAACAGCTTCCGGAGAAATACAAAGAAATATTCTTCGACCAGTCCAAGAA
TGGTTACGCGGGCTACATCGATGGTGGTGCATCACAGGAAGAGTTCTATA
AATTTATTAAACCAATCCTTGAGAAAATGGATGGCACGGAAGAGTTACTT
GTTAAACTTAACCGCGAAGACTTGCTTAGAAAGCAACGTACATTCGACAA
CGGCTCCATCCCACACCAGATTCATTTAGGTGAACTTCACGCCATCTTGC
GCAGACAAGAAGATTTCTATCCCTTCTTAAAAGACAATCGGGAGAAAATC
GAGAAGATCCTGACGTTCCGCATTCCCTATTATGTCGGTCCCCTGGCACG
TGGTAATTCTCGGTTTGCCTGGATGACGCGCAAAAGTGAGGAAACCATCA
CCCCTTGGAACTTTGAAGAAGTCGTGGATAAAGGTGCTAGCGCGCAGTCT
TTTATAGAAAGAATGACGAACTTCGATAAAAACTTGCCCAACGAAAAAGT
CCTGCCCAAGCACTCTCTTTTATATGAGTACTTTACTGTGTACAACGAAC
TGACTAAAGTGAAATACGTTACGGAAGGTATGCGCAAACCTGCCTTTCTT
AGTGGCGAGCAGAAAAAAGCAATTGTCGATCTTCTCTTTAAAACGAATCG
CAAGGTAACTGTAAAACAGCTGAAGGAAGATTATTTCAAAAAGATCGAAT
GCTTTGATTCTGTCGAGATCTCGGGTGTCGAAGATCGTTTCAACGCTTCC
TTAGGGACCTATCATGATTTGCTGAAGATAATAAAAGACAAAGACTTTCT
CGACAATGAAGAAAATGAAGATATTCTGGAGGATATTGTTTTGACCTTGA
CCTTATTCGAAGATAGAGAGATGATCGAGGAGCGCTTAAAAACCTATGCC
CACCTGTTTGATGACAAAGTCATGAAGCAATTAAAGCGCCGCAGATATAC
GGGGTGGGGCCGCTTGAGCCGCAAGTTGATTAACGGTATTAGAGACAAGC
AGAGCGGAAAAACTATCCTGGATTTCCTCAAATCTGACGGATTTGCGAAC
CGCAATTTTATGCAGCTTATACATGATGATTCGCTTACATTCAAAGAGGA
TATTCAGAAGGCTCAGGTGTCTGGGCAAGGTGATTCACTCCACGAACATA
TAGCAAATTTGGCCGGCTCTCCTGCGATTAAGAAGGGGATCCTGCAAACA
GTTAAAGTTGTGGATGAACTTGTAAAAGTAATGGGCCGCCACAAGCCGGA
GAATATCGTGATAGAAATGGCGCGCGAGAATCAAACGACACAAAAAGGTC
AAAAGAACTCAAGAGAGAGAATGAAGCGCATTGAGGAGGGGATAAAGGAA
CTTGGATCTCAAATTCTGAAAGAACATCCAGTTGAAAACACTCAGCTGCA
AAATGAAAAATTGTACCTGTACTACCTGCAGAATGGAAGAGACATGTACG
TGGATCAGGAATTGGATATCAATAGACTCTCGGACTATGACGTAGATCAC
ATTGTCCCTCAGAGCTTCCTCAAGGATGATTCTATAGATAATAAAGTACT
TACGAGATCGGACAAAAATCGCGGTAAATCGGATAACGTCCCATCGGAGG
AAGTCGTTAAAAAGATGAAAAACTATTGGCGTCAACTGCTGAACGCCAAG
CTGATCACACAGCGTAAGTTTGATAATCTGACTAAAGCCGAACGCGGTGG
TCTTAGTGAACTCGATAAAGCAGGATTTATAAAACGGCAGTTAGTAGAAA
CGCGCCAAATTACGAAACACGTGGCTCAGATCCTCGATTCTAGAATGAAT
ACAAAGTACGATGAAAACGATAAACTGATCCGTGAAGTAAAAGTCATTAC
CTTAAAATCTAAACTTGTGTCCGATTTCCGCAAAGATTTTCAGTTTTACA
AGGTCCGGGAAATCAATAACTATCACCATGCACATGATGCATATTTAAAT
GCGGTTGTAGCACGGCCCTTATTAAGAAATACCCTAAACTCGAAAGTGAG
TTTGTTTATGGGGATTATAAAGTGTATGACGTTCGCAAAATGATCGCGAA
ATCAGAACAGGAAATCGGTAAGGCTACCGCTAAATACTTTTTTTATTCCA
ACATTATGAATTTTTTTAAGACCGAAATAACTCTCGCGAATGGTGAAATC
CGTAAACGGCCTCTTATAGAAACCAATGGTGAAACGGGAGAAATCGTTTG
GGATAAAGGTCGTGACTTTGCCACCGTTCGTAAAGTCCTCTCAATGCCGC
AAGTTAACATTGTCAAGAAGACGGAAGTTCAAACAGGGGGATTCTCCAAA
GAATCTATCCTGCCGAAGCGTAACAGTGATAAACTTATTGCCAGAAAAAA
AGATTGGGATCCAAAAAAATACGGAGGCTTTGATTCCCCTACCGTCGCGT
ATAGTGTGCTGGTGGTTGCTAAAGTCGAGAAAGGGAAAAGCAAGAAATTG
AAATCAGTTAAAGAACTGCTGGGTATTACAATTATGGAAAGATCGTCCTT
TGAGAAAAATCCGATCGACTTTTTAGAGGCCAAGGGGTATAAGGAAGTGA
AAAAAGATCTCATCATCAAATTACCGAAGTATAGTCTTTTTGAGCTGGAA
AACGGCAGAAAAAGAATGCTGGCCTCCGCGGGCGAGTTACAGAAGGGAAA
TGAGCTGGCGCTGCCTTCCAAATATGTTAATTTTCTGTACCTTGCCAGTC
ATTATGAGAAACTGAAGGGCAGCCCCGAAGATAACGAACAGAAACAATTA
TTCGTGGAACAGCATAAGCACTATTTAGATGAAATTATAGAGCAAATTAG
TGAATTTTCTAAGCGCGTTATCCTCGCGGATGCTAATTTAGACAAAGTAC
TGTCAGCTTATAATAAACATCGGGATAAGCCGATTAGAGAACAGGCCGAA
AATATCATTCATTTGTTTACCTTAACCAACCTTGGAGCACCAGCTGCCTT
CAAATATTTCGATACCACAATTGATCGTAAACGGTATACAAGTACAAAAG
AAGTCTTGGACGCAACCCTCATTCATCAATCTATTACTGGATTATATGAG
ACACGCATTGATCTTTCACAGCTGGGCGGAGACAAGAAGAAAAAACTGAA
ACTGCACCATCATCACCATCATCATCACCATCATTGATAACTCGAG

The ligation mix was then used to transform Bacillus subtilis C2987 cells and about 100 transformants were obtained. Eight (8) colonies were picked, and their sequences were confirmed after mini-prep. Those were then pooled and used to transform CB20-1 and Bacillus subtilis 168 cells.

Two transformants of Spy-Cas9 (A and B) were picked and grown in individual 2-mL pre-cultures made with LB and 10 ppm neomycin. A volume of 1 mL of the pre-culture was used to inoculate 35 mL of Grant's II Medium with 10 ppm neomycin. The cultures were then grown for about 63 hours at 37° C., shaking at 280 rpm, and maintained at 70% humidity in Ultra-Yield Flasks using enhanced seals.

After the cultures are completed, the broths were centrifuged and the cell pellets and supernatants were stored separately. A cell pellet was taken from 1 mL of cells out of each of the cultures, and the pellets were suspended in 0.5 mL of Buffer P1. Five (5) mL of Ready-Lyse (a T4 lysozyme) was then added to each mixture. The mixtures were incubated at 37° C. for about 0.5 hour.

The cultures were observed to become viscous at the end of 0.5 hour. An Omnicleave nuclease at the volume of 5 mL was added and the incubation was carried on for another 0.5 hour. While the samples were still turbid, the mixture has reduced viscosity as a result of lysis. The lysed cell pellets were then put onto SDS-PAGE and His-Tag detection was carried out using Western Blots. Expression of SpyCas9 was observed.

2. Targeting and Editing the phrA gene of Bacillus Subtilis

The sequence of the phrA gene, which is involved in the early sporulation pathway of Bacillus subtilis, is presented below as SEQ ID NO: 27, with the targeting site underlined.

(SEQ ID NO: 27)
ATGAAATCTAAATGGATGTCAGGTTTGTTGCTCGTTGCGGTCGGGTTCAG
CTTTACTCAGGTGATGGTTCTACTTTAGATTTACCTACAGTCCAAACAAC
GAGCAACGCCAGCCCAAGTCGAAATGAGTCCACTACCAAGATGCAGGTGA
AACAGCAAACACAGAAGGGAAAACATTTCATATTGCGGCACGCAATCAAA
CATGATACGTCCACTTTGTCGTTTGTGTCTTCCCTTTTGTAAAGTATAAC
GCCGTGCGTTAGTTTGTACT

Deletion of the phrA gene was expected to cause uncontrolled RapA phosphatase activity with the consequence of non-initiation of sporulation (see, Proc. Natl. Acad. Sci. USA, 94 (1997): 8612-8616).

The targeting sequence, underlined above and presented herein as SEQ ID NO: 28, included the PAM site “AGG”, was used as the target site for Cas9 activity. A T7 promoter was added preceding the 20-bp phrA sequence without the PAM site nucleotides, and the guide scaffold sequence was added to the 3′ end. The resulting sequence was used as a template for in vitro guide RNA synthesis, applying the MegaShort Script Kit (Ambion) and the RNAEasy kit (Qiagen), which was used for guide RNA purification.

A wild type Bacillus subtilis strain 168 (trpC2) was obtained from the Bacillus Genetic Stock Center. A transformation mixture comprising the Cas9 expression plasmid with 2 ultramers (154-base single stranded upper and lower strand oligonucleotides containing the entire phrA open reading frame having a 19-base stop codon insertion), and the in vitro synthesized guide RNA as described above, was then grown for about 30 hours at 37° C. in Schaeffer's sporulation medium.

The control strains without the Cas9 expression plasmid were compared with the strains expressing Cas9. The percentage of sporulation of Cas9 and non-Cas9 was calculated and presented as ratios of spore counts versus viable cell counts. It was observed that sporulation was abolished in the Cas9 strains, which indicated the disruption of the phrA gene.

The primer pairs of SEQ ID NO: 29 (forward primer) and SEQ ID NO: 30 (reverse primer) were used for colony PCR.

PCR amplification of the phrA gene and subsequent restriction digestion using PmeI or PacI showed a double band of about 70-80 bases on a 4% agarose gel. This indicated that homologous directed repair of the phrA gene using the donor signal strand oligonucleotides was achieved.

### Example 10

### Expression of Streptococcus Pyogenes CAS9 in Streptomyces Lividans

In the literature Cas9-mediated, targeted chromosomal deletions were reported in three different types of Streptomyces strains with varied efficiencies ranging from 70 to 100% (See, ACS Synthetic Biology (2015) 19:4(6) 723-728). This reported work used donor templates of several kilobases in length. The gene encoding S. pyogenes Cas9, codon optimized for expression in Streptomyces lividans is set forth in SEQ ID NO: 31.

In the present experiment, donor templates including ultramers in the form of single strand oligonucleotides were designed to effect Cas9-mediated gene editing in Streptomyces. Specifically, CRISPR-Cas9 can be used to delete two Streptomyces genes, sco7700 and sco7701, which belong to a two-gene operon responsible for methylisoborneol (MIB) biosynthesis. Methylisoborneol is a volatile organic compound produced by Streptomyces, which is thought to be responsible for the characteristic smell of moist soil as well as a number of unpleasant tastes or odors that is often deemed undesirable or even problematic in large scale fermentation plants. See, J. Am. Chem. Soc. (2008) 16: 130(28):8908-8909.

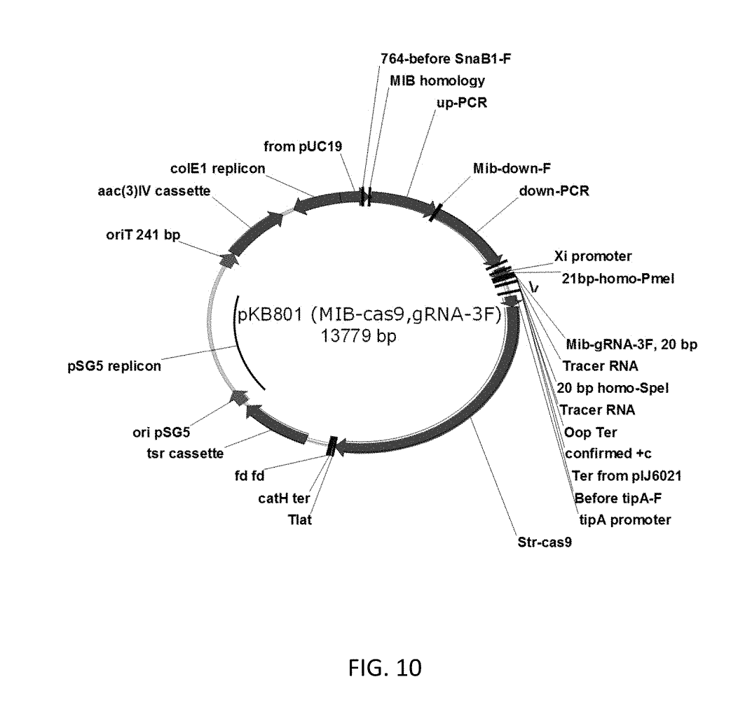

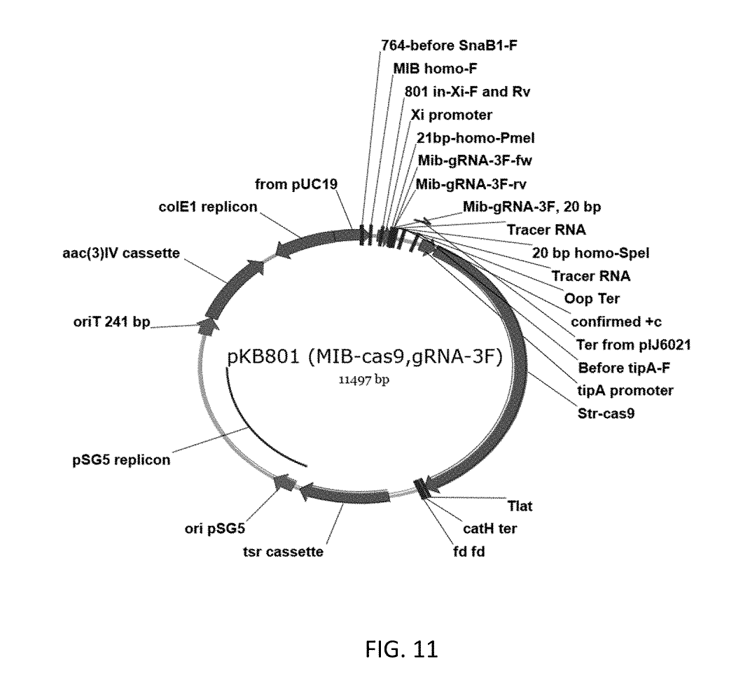

FIG. 10 depicts the expression cassette with the Cas9 gene and the guide RNA sequence together with the 20 bp target site of SEQ ID NO: 32, with the 2 kb homology repair donor in a plasmid as control. FIG. 11 depicts the expression cassette without the 2 kb homology repair donor template, in order to allow for the use of 200-base ultramers with stop codons.

Two (2) μg of the Cas9-gRNA expression cassette and 100 μM of ultramers of 200 bases oligonucleotides (SEQ ID NO: 33) was used to disrupt the sco7700 and sco7701 genes.

Disruption of the MIB genes was confirmed using the absence of odor from a 50 mL culture cultivated at 30° C. PCR amplification of the MIB gene, followed by Pme1 or Pac1 restriction digestion further, more precisely, verified the disruption of the MIB gene.

### REFERENCES

PCT Publication No. WO 1991/17243
PCT Publication No. WO 2013/141680
PCT International Application No. PCT/CN2014/093918
U.S. Patent Application Publication No. 2011/0020899
U.S. Patent Application Publication No. 2013/0323798
U.S. Pat. No. 5,107,065
U.S. Pat. No. 6,022,725
U.S. Pat. No. 8,697,359
Bleuyard et al., DNA Repair 5:1-12, 2006.
ACS Synthetic Biology (2015) 19:4(6) 723-8
J. Am. Chem. Soc. (2008) 16: 130(28):8908-9.
Sambrook et al., MOLECULAR CLONING: A LABORATORY MANUAL, 2^{nd} ed., Cold Spring Harbor, 1989, and 3rd ed., 2001.
Cong et al., Science, 339:819-823, 2013.
Siebert and Puchta, Plant Cell 14:1121-1131, 2002.
Fonfara et al., Nucleic Acids Res., pages 1-14, 2013.
Pacher et al., Genetics 175:21-29, 2007.
Cao et al., Science 9:991-1001, 2000.
Campbell et al., Curr. Genet. 16: 53-56, 1989.
Fonfara et al., Nucleic Acids Res., pages 1-14, 2013.
Hawksworth et al., “Ainsworth and Bisby's Dictionary of The Fungi”, 8th edition, 1995, CAB International, University Press, Cambridge, UK.
Henikoff and Henikoff, Proc. Natl. Acad. Sci. USA 89:10915, 1989.
Higgins and Sharp, CABIOS 5:151-153, 1989.
Higgins et al., Comput. Appl. Biosci. 8:189-191, 1992.
Hsu et al., “Development and Applications of CRISPR-Cas9 for Genome Engineering”, Cell 157: 1262-1278, 2014.
Liu et al., “Efficient genome editing in filamentous fungus Trichoderma reesei using the CRISPR/Cas9 system”, Cell Discovery, Vol. 1 (No. 15007), 2015.
Liu et al., Acta Biochim. Biophys. Sin (Shanghai) 40(2): 158-165, 2008.
Needleman and Wunsch, J. Mol. Biol. 48:443-453, 1970.
Nikolaev et al., Biotechnol. J. (8):905-911, 2013.
Rudin et al., “Genetic and Physical Analysis of Double-Strand Break Repair and Recombination in Saccharomyces Cerevisiae”, Genetics 122:519-534, 1989.
Smith et al., Nucl. Acids Res. 23:5012-5019.

1. A method for genome editing in a filamentous fungal cell, the method comprising introducing into the filamentous fungal cell a Cas endonuclease, a guide polynucleotide, and a donor polynucleotide, wherein the donor polynucleotide comprises at least one homology arm, wherein the homology arm is less than 500 nucleotides in length and comprises sequence homology to a targeted genomic locus of the fungal cell, wherein the Cas endonuclease and guide polynucleotide form a complex that enables the Cas endonuclease to act at or near the targeted genomic locus of the fungal cell.

2. The method of claim 1, wherein the donor polynucleotide is inserted into the targeted genomic locus of the fungal cell.

3. The method of claim 1, wherein the donor polynucleotide further comprises a nucleotide sequence of interest which is either upstream (5′) and operably linked to the homology arm or downstream (3′) and operably linked to the homology arm.

4. The method of claim 3, wherein the nucleotide sequence of interest is inserted into the targeted genomic locus of the fungal cell.

5. The method of claim 2, wherein the inserted donor polynucleotide comprises a genome modification selected from the group consisting of a DNA deletion, a DNA disruption, a DNA insertion, a DNA inversion, a DNA point mutation, a DNA replacement, a DNA knock-in, a DNA knock-out and a DNA knock-down.

6. The method of claim 4, wherein the inserted nucleotide sequence of interest comprises a genome modification selected from the group consisting of a DNA deletion, a DNA disruption, a DNA insertion, a DNA inversion, a DNA point mutation, a DNA replacement, a DNA knock-in, a DNA knock-out and a DNA knock-down.

7. The method of claim 1, wherein the homology arm is less than 350 nucleotides in length.

8. The method of claim 1, wherein the homology arm is less than 150 nucleotides in length.

9. The method of claim 1, wherein the homology arm is between 100-40 nucleotides in length.

10. The method of claim 1, wherein the donor polynucleotide comprises a homology arm upstream (5′) and operably linked to a nucleotide sequence of interest and a homology arm downstream (3′) and operably linked to the same nucleotide sequence of interest, wherein at least one of the two homology arms are less than 500 nucleotides in length.

11. The method of claim 10, wherein the nucleotide sequence of interest is inserted into the targeted genomic locus of the fungal cell.

12. The method of claim 10, wherein at least one homology arm is less than 350 nucleotides in length.

13. The method of claim 10, wherein at least one homology arm is less than 150 nucleotides in length.

14. The method of claim 10, wherein at least one homology arm is between 100-40 nucleotides in length

15. The method of claim 10, wherein both homology arms are less than 500 nucleotides.

16. The method of claim 3, wherein the nucleotide sequence of interest comprises at least one heterologous nucleotide.

17. The method of claim 3, wherein the nucleotide sequence of interest comprises a heterologous polynucleotide sequence.

18. The method of claim 10, wherein the nucleotide sequence of interest comprises at least one heterologous nucleotide.

19. The method of claim 10, wherein the nucleotide sequence of interest comprises a heterologous polynucleotide sequence.

20. The method of claim 1, wherein the Cas endonuclease is a Cas nickase or a functional variant thereof.

21. The method of claim 1, wherein the Cas endonuclease is a Cas9 endonuclease or a functional variant thereof.

22. The method of claim 21, wherein the Cas9 endonuclease is a species selected from the group consisting of Streptococcus sp., Campylobacter sp., Neisseria sp., Francisella sp. and Pasteurella sp.

23. The method of claim 1, wherein the introducing step comprises introducing a polynucleotide construct comprising an expression cassette for expressing the Cas endonuclease or a functional variant thereof in the fungal cell.

24. The method of claim 1, wherein the introducing step comprises introducing a polynucleotide construct comprising an expression cassette for expressing the guide RNA in the fungal cell.

25. The method of claim 1, wherein the introducing step comprises introducing into the fungal cell a circular polynucleotide construct comprising an expression cassette for the Cas endonuclease, an expression cassette for the guide RNA, and the donor DNA.

26. The method of claim 1, wherein the introducing step comprises directly introducing the guide polynucleotide or Cas endonuclease into the fungal cell.

27. The method of claim 1, wherein the Cas endonuclease or a functional variant thereof is operably linked to a nuclear localization signal.

28. The method of claim 1, wherein the donor polynucleotide is a double strand DNA.

29. The method of claim 1, wherein the donor polynucleotide is a single strand DNA.

30. The method of claim 1, wherein the filamentous fungal cell is selected from the genus consisting of Trichoderma, Penicillium, Aspergillus, Humicola, Chrysosporium, Fusarium, Myceliophthora, Neurospora and Emericella.

31. A recombinant filamentous fungal cell produced by the method of claim 1.
